(19) United States
(12) Patent Application Publication
(10) Pub. No.: US 2022/0012766 A1
(43) Pub. Date: Jan. 13, 2022

(54) METHODS AND SYSTEMS FOR FACILITATING MANAGING PURCHASE OF MEDICATIONS

(71) Applicant: Benjamin Samuel Hinton, II, Gastonia, NC (US)

(72) Inventor: Benjamin Samuel Hinton, II, Gastonia, NC (US)

(21) Appl. No.: 17/373,572

(22) Filed: Jul. 12, 2021

Related U.S. Application Data
(60) Provisional application No. 63/050,681, filed on Jul. 10, 2020.

(51) Int. Cl. G06Q 30/02; G16H 20/10; G06Q 30/06

(52) U.S. Cl. CPC G06Q 30/0239; G06Q 30/0607; G16H 20/10; G06Q 30/0201

(57) ABSTRACT

Disclosed herein is a method for facilitating managing purchase of medications. Accordingly, the method may include receiving a medication request data associated with a patient from a device, identifying stores based on the medication description, receiving prices offered by the stores for the medication from store devices associated with the stores, retrieving a discount card data of a discount card associated with the patient based on an patient identifier, applying the discount card data to the prices offered by the plurality of stores for the medication, generating discounted prices offered by the stores for the medication based on the applying, retrieving store data associated with the stores based on the identifying, and transmitting the discounted prices offered by the stores for the medication, the discount card data, and the store data to the device.

The current application claims a priority to the U.S. Provisional Patent application serial number 63/050,681 filed on Jul. 10, 2020. The current application is filed on Jul. 12, 2021, whereas Jul. 10, 2021 and Jul. 11, 2021 were on a weekend.

### FIELD OF THE INVENTION

Generally, the present disclosure relates to the field of data processing. More specifically, the present disclosure relates to methods and systems for facilitating managing purchase of medications.

### BACKGROUND OF THE INVENTION

The field of data processing is technologically important to several industries, business organizations, and/or individuals.

Since the early 1980s, spending on drugs has increased at nearly 4 percent annually, even after adjusting for general inflation and population growth. Prescription drug expenditures in the United States are currently about 17 percent of the overall cost of personal health care services (Kesselheim et al., 2016) Prescription drug spending increased 5.7% to $369.7 billion in 2019, faster than the 3.8% growth in 2018. Millions of uninsured and underinsured Americans struggle to afford the medicines they need, even forgoing medically necessary drugs when prices are out of reach. When discussing the high cost of prescription drugs, politicians often focus on the financial burden carried by senior citizens. Unfortunately, high prescription drug prices are a problem for Americans of all ages, particularly for the uninsured. According to the CBO, the number of American citizens who are uninsured in 2020 is around 31 million. This number is expected to increase significantly due to the changing mindset of the population. In present times, the individuals who are uninsured or underinsured, as well as those who are insured are always looking for ways to decrease drug costs and compare the prices of medications. Medication prices have done nothing but skyrocket over the past few years, as Americans spend more on medications than any other country in the entire world. The prices are inflating at a faster rate than many other things we buy. Even with insurance, in addition to a monthly premium, a lot of patients are still coming out of their pocket for a high co-pay. In some cases, the cost is more through insurance than if they were to pay the cash price. In addition, often the individual's PBM or health plan may require a patient to go to a specific chain pharmacy or their own provider's pharmacy, which may not be in the best interest of the patient. As a result, the patient must choose between service and their medications not being covered, which undoubtedly causes a spike in their medication prices, as they will otherwise not be covered by their insurance plan. If a patient is uninsured, the situation can be more of a burden, because without insurance the price of the medication is as high as it will ever be, which leads to the business of prescription discount cards, which as a result has been a growing market. However, even then at times, the cash price is cheaper without a discount card than with one applied due to the mark-up in drug prices between the pharmacy benefit management (PBM) and pharmacies.

Furthermore, existing retail computer processing systems only allow a person to process the price of one prescription drug at one time which results in a lot of time spent from both the pharmacy and the patient having to type in the prescription details of the drug into each discount site individually if the patient is trying to compare multiple discount cards to obtain the lowest price. Patients may be able to compare the prices of a particular drug through existing apps between pharmacies, however, the ability of an app to compare discount card prices and cross reference them with data listing is unheard of. As a result, This ends up being a hassle not only for pharmacy, but also for the patient, caregivers, and consumers involved. When a person has multiple discount cards they wish to try to process their prescription with, they generally have no idea of which would be most cost-effective for the particular drug, and going through the motions of doing so would potentially slow down pharmacy production and lengthens lines, which end up in longer wait times, decreased customer satisfaction and most importantly more time patients are going without their medication that they already have to pay for monetarily. In addition, there is also a discrepancy because the price of medication differs at different locations. Data shows that even though the existing technologies may show a certain price of a drug for a certain chain of pharmacies, the actual price depends on the pharmacy store location itself, which potentially frustrates both the consumer and the pharmacy staff at the time of billing and point of sale. Therefore, in the event a patient is in an emergency or a severe medical crisis, the situation could lead to the patient having to settle for the first price they're presented at the pharmacy that is better than the cash price or the copay listed under insurance, instead of obtaining the lowest price available. With a record $67 billion being paid out-of-pocket by patients for all prescriptions filled in retail pharmacies in 2019, up from $61 billion in 2015, there is a lot of cause for concern in the trend, and supportive measures need to be taken to help patients offset some of the drug costs.

Opting out of purchasing and taking medications can open the door up to serious health risks. There is a lot of growing concern around patients abandoning their new prescriptions and not following a physician's recommendation for care. Abandonment occurs much more often when the patients have higher out-of-pocket costs. In 2019, 9% of all new prescription starts were abandoned at retail pharmacies, representing patient care recommended by a physician but not received by the patient. While abandonment rates are less than 5% when the prescription carries no out-of-pocket cost, it rises to 45% when the cost is over $125 and 60% when the cost is over $500. For many uninsured, those rising costs mean they have to choose between taking the medication they need and paying for necessities like rent and food. In fact, a 2016 survey found that 14% of uninsured Americans had either skipped doses or didn't fill a prescription because of the cost. In short: Drug prices are costing Americans more than just dollars—they're costing our health, too. Even for those patients with insurance, it has been reported that the prices of medications without insurance may be cheaper 25% of the time. The question still remains, how else will a patient know what the lowest cost of their medication could be? Further, existing technologies do not allow the patients to view the price of multiple prescription drug cards at the same time to solidify the cheapest price of the medication is found without having to submit the drug information into each prescription drug card program for an individual price.

Most patients are unaware of what a prescription savings card is, let alone how to search for more than one. They expect the pharmacy technician to provide them with the lowest price by searching through each of these, which potentially causes a disconnect in the fast-paced atmosphere when the information is repeatedly typed in the system in the form of a guessing game to obtain the biggest discount per the patient's request. The variables for obtaining the lowest discount card price include which pharmacy the patient plans to go to, the location, the chain, the medication, and the quantity., all of which the patient has to try to figure out to find the most affordable price.

The high cost of health care is a burden on individual patients, their families, and society as a whole. People with chronic health conditions are particularly vulnerable because their illnesses or the treatments for their illnesses impede their ability to work, with some patients losing employment altogether. Such individuals frequently incur significant financial debt and deplete the assets they need to pay for treatment, some to the extent that they must resort to bankruptcy. Cancer patients especially face severe financial risks—or “financial toxicity” (NCI, 2017)—and have a materially higher rate of personal bankruptcy than those who have not been diagnosed with cancer (Ramsey et al., 2013). Further, the prices of medications are steadily increasing and many consumers and patients with or without insurance are being affected. Non-compliance is often a result of not being able to afford their medications. It seems to be a never-ending cycle. The patients have a medical problem, so they see a physician where they're prescribed medication. Based on the price, the patients may not be able to afford to even pick up the medication for the first time, not to mention refills if it's a chronic disease. Therefore, time, money, effort, and gas are used up in an attempt to “shop around town” for the lowest drug price not only between pharmacies but between the various prescription drug cards. If patients are loyal and enjoy the service at a particular pharmacy, they will often continue to pay an increased cost if they choose not to explore other options. If one particular pharmacy tends to use one specific discount card, the patient may not ever receive the savings that they are entitled to. It's also important to settle on a pharmacy that consistently offers good deals on medication and fills all your prescriptions there because that makes it easier for pharmacists to spot potentially dangerous interactions and other safety concerns. When jumping from pharmacy to pharmacy if all the drugs aren't transferred from your profile, the pharmacist won't know how to counsel against these interactions. Further, existing technologies do not determine these discounts at a consistent pharmacy.

In most cases, the price of the medication is cheaper with insurance, but in many cases, it's also cheaper without insurance using a “drug card” instead. The population most affected are those within the “donut hole” in their plans and those who simply can't afford to not have any insurance at all. 9 percent of U.S. adults, or roughly 28 million people, who don't have health insurance and must pay all of their drug costs and other healthcare expenses, says Orly Avitzur, M.D., Consumer Reports' medical director. But it can be worth the effort even when the patient is insured. Some of the lowest retail prices in pharmacies may sometimes be a better deal than using insurance, especially in the case of drugs that aren't covered well. Yet many people seem unaware of how effective shopping around can be. In a recent CR survey, only 22 percent of current prescription drug takers who had paid more for their medication in the previous year said they comparison shopped for a better deal. Therefore, the problem then lies in finding exactly which discount card can help them pay for their medication, as all prescription drug cards claim to offer huge savings. The claims range from saving up to 90% to no copays. There are always well-known discount cards names such as GoodRx, Blink Health, or Singlecare. However, existing technologies do not determine if the discount is the lowest price as the discount cards offer. Unfortunately, every day patients take the price at face value and dig deep in their wallets and pockets and pay for their prescriptions, sacrificing in other areas of their lives. When more than half of all people in the United States routinely use prescription drugs, and 15 percent of the population regularly takes five or more drugs (Kantor et al., 2015), there is a lot of room for opportunity in assisting patients to medicine shopping efforts to obtain a good price for their medications.

The general public considers lowering the cost of prescription drugs as one of its highest health care priorities. In 2017, 61 percent of the respondents in a poll assigned the issue a top priority (KFF, 2017a), with another survey ranking it first among domestic issues requiring priority action from the U.S. Congress (Politico, 2017). Although the vast majority of the public believes that prescription drugs have improved the lives of people in the United States, most also have an unfavorable view of pharmaceutical companies and believe that they make excessive profits (KFF, 2017b). Not surprisingly, patients who take multiple prescriptions, as well as low-income and uninsured patients, are most likely to report having difficulty affording prescription medications (KFF, 2017b).

As a consumer or patient, comparing or evaluating the different card program opportunities for drug discounts is especially difficult because there is no standard reference price or discounting method, not to mention there are so many out there, all claiming to do the same thing. There isn't one central percentage that people can save per medication or savings card. Presently, different patients pay different prices for identical drugs, with individual prices depending mainly on the specifics of their health insurance plans, which generally include cost-sharing features such as copays, deductibles, and coinsurance. In addition, uninsured patients suffer these same shortcomings in terms of the differing price from pharmacy to pharmacy, and between discount cards. In severe financial circumstances, patients' health care expenses also adversely affect other members of their families The challenge is that savings and prices also vary depending on the medication, strength, amount, and the actual drug store/method the patient uses to acquire their medication. The most favorable option for any individual ultimately is chosen based on the evaluation of these combined factors. As a result, in terms of using a prescription discount card, a single savings card that has the best price for each medication is often unlikely. In the end, if the consumer does not wish to put in additional effort to try and research and try more discount cards in an attempt to bring down the price, the patient may just settle paying for whatever the price was under the original discount card used for the previous medication, or whichever card is at hand at the time and processed by the pharmacy or even simply paying the inflated cash price. Another frequent problem that consumers run into includes the limited pricing quotes to a subset of available drugs. Different pharmacies use different generic drugs, and the prices sometimes vary based on the specific one that they have in stock. The discounts that are expressed on the cards act like an average from undisclosed prices, that of which the consumer will never see because that's how the PBMs, pharmacies, and drug manufacturers make their profits.

Until recently, some insurance companies could prohibit pharmacies from disclosing the cash prices of medications, or the cost without applying for insurance. This struggle has been going on for quite a while now, as according to a report by Kaiser Family Foundation back in February 2002, from the consumer's perspective these prescription discount programs have the potential to offer substantial savings, however, the plethora of discount card options presents a formidable landscape to navigate. Comparison shopping among these options can be tedious and confusing. It's difficult to keep up with the prices that one card may offer because they can change each month, let alone contrasting the price against another one. In fact, for consumers who typically fill prescriptions only when experiencing an acute condition, comparison-shopping is probably not feasible. Even in patients with chronic conditions, oftentimes the patients are confused when the prices shift due to new contracts within PBMs and pharmacies, etc. The response may be out of confusion because the price for their medication this month isn't what the patients were charged the last month even though it may have been billed the same way. Further, the patients may also place a higher priority on accessing the prescription immediately and forego the deeper discounts that may be available from mail-order services and other methods because of the time delay in shipping. Consumers lacking access to the internet are even more disadvantaged in their efforts to evaluate card options. Often those who are underserved are also the main populations who aren't insured, and as a result, depending on discount cards to reduce the prices.

Therefore, there is a need for improved methods and systems for facilitating managing purchase of medications that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method for facilitating managing purchase of medications, in accordance with some embodiments. Accordingly, the method may include receiving, using a communication device, at least one medication request data associated with a patient from at least one device. Further, the at least one medication request data may include at least one medication description of at least one medication and at least one patient identifier of the patient. Further, the method may include identifying, using a processing device, a plurality of stores based on the at least one medication description. Further, the method may include receiving, using the communication device, a plurality of prices offered by the plurality of stores for the at least one medication from a plurality of store devices associated with the plurality of stores. Further, the method may include retrieving, using a storage device, at least one discount card data of at least one discount card associated with the patient based on the at least one patient identifier. Further, the method may include applying, using the processing device, the at least one discount card data to the plurality of prices offered by the plurality of stores for the at least one medication. Further, the method may include generating, using the processing device, a plurality of discounted prices offered by the plurality of stores for the at least one medication based on the applying. Further, the method may include retrieving, using the storage device, a plurality of store data associated with the plurality of stores based on the identifying. Further, the method may include transmitting, using the communication device, the plurality of discounted prices offered by the plurality of stores for the at least one medication, the at least one discount card data, and the plurality of store data to the at least one device.

Further disclosed herein is a system for facilitating managing purchase of medications. Accordingly, the system may include a communication device configured for receiving at least one medication request data associated with a patient from at least one device. Further, the at least one medication request data may include at least one medication description of at least one medication and at least one patient identifier of the patient. Further, the communication device may be configured for receiving a plurality of prices offered by a plurality of stores for the at least one medication from a plurality of store devices associated with the plurality of stores. Further, the communication device may be configured for transmitting a plurality of discounted prices offered by the plurality of stores for the at least one medication, at least one discount card data, and a plurality of store data to the at least one device. Further, the system may include a processing device communicatively coupled with the communication device. Further, the processing device may be configured for identifying the plurality of stores based on the at least one medication description. Further, the processing device may be configured for applying the at least one discount card data to the plurality of prices offered by the plurality of stores for the at least one medication. Further, the processing device may be configured for generating the plurality of discounted prices offered by the plurality of stores for the at least one medication based on the applying. Further, the system may include a storage device communicatively coupled with the processing device. Further, the storage device may be configured for retrieving the at least one discount card data of at least one discount card associated with the patient based on the at least one patient identifier. Further, the storage device may be configured for retrieving the plurality of store data associated with the plurality of stores based on the identifying.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating managing purchase of medications, in accordance with some embodiments.

FIG. 3 is a flowchart of a method for facilitating managing purchase of medications, in accordance with some embodiments.

FIG. 4 is a flowchart of a method for selecting at least one store for facilitating managing the purchase of medications, in accordance with some embodiments.

FIG. 5 is a flowchart of a method for generating at least one navigational direction for facilitating managing the purchase of medications, in accordance with some embodiments.

FIG. 6 is a flowchart of a method for identifying a store offering the lowest discounted price for facilitating managing the purchase of medications, in accordance with some embodiments.

FIG. 7 is a flowchart of a method for receiving the at least one discount card data for facilitating managing the purchase of medications, in accordance with some embodiments.

FIG. 8 is a flowchart of a method for determining at least one discounted price for facilitating managing the purchase of medications, in accordance with some embodiments.

FIG. 9 is a flowchart of a method for generating an order reference for facilitating managing the purchase of medications, in accordance with some embodiments.

FIG. 10 is a screenshot of a user interface associated with a software application for facilitating managing purchase of medications, in accordance with some embodiments.

FIG. 11 is a screenshot of a user interface associated with the software application, in accordance with some embodiments.

FIG. 12 is a screenshot of a user interface associated with the software application, in accordance with some embodiments.

FIG. 13 is a screenshot of a user interface associated with the software application, in accordance with some embodiments.

FIG. 14 is a screenshot of a user interface associated with the software application, in accordance with some embodiments.

FIG. 15 is a screenshot of a user interface associated with the software application, in accordance with some embodiments.

FIG. 16 is a screenshot of a user interface associated with the software application, in accordance with some embodiments.

FIG. 17 is a screenshot of a user interface associated with the software application, in accordance with some embodiments.

FIG. 18 is a screenshot of a user interface associated with the software application, in accordance with some embodiments.

FIG. 19 is a screenshot of a user interface associated with the software application, in accordance with some embodiments.

FIG. 20 is a screenshot of a user interface associated with the software application, in accordance with some embodiments.

FIG. 21 is a screenshot of a user interface associated with the software application, in accordance with some embodiments.

FIG. 22 is a screenshot of a user interface associated with the software application, in accordance with some embodiments.

FIG. 23 is a screenshot of a user interface associated with the software application, in accordance with some embodiments.

FIG. 24 is a screenshot of a user interface associated with the software application, in accordance with some embodiments.

FIG. 25 is a screenshot of a user interface associated with the software application, in accordance with some embodiments.

FIG. 26 is a flow diagram of a method for facilitating comparing prices of medications between different drug discount cards, in accordance with some embodiments.

FIG. 27 is a flow diagram of a method for facilitating comparing prices of medications between different drug discount cards, in accordance with some embodiments.

FIG. 28 is a flow diagram of a method for facilitating comparing prices of medications between different drug discount cards, in accordance with some embodiments.

FIG. 29 is a flow diagram of a method for facilitating comparing prices of medications between different drug discount cards, in accordance with some embodiments.

FIG. 30 is a flow diagram of a method for facilitating comparing prices of medications between different drug discount cards, in accordance with some embodiments.

FIG. 31 is a flow diagram of a method for facilitating comparing prices of medications between different drug discount cards, in accordance with some embodiments.

FIG. 32 is a flow diagram of a method for facilitating comparing prices of medications between different drug discount cards, in accordance with some embodiments.

FIG. 33 is a flow diagram of a method for facilitating comparing prices of medications between different drug discount cards, in accordance with some embodiments.

FIG. 34 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems for facilitating managing purchase of medications, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), and a biometric sensor (e.g. a fingerprint sensor), etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes methods and systems for facilitating managing purchase of medications. Further, the disclosed system may be configured for comparing the prices of medications between different drug discount cards.

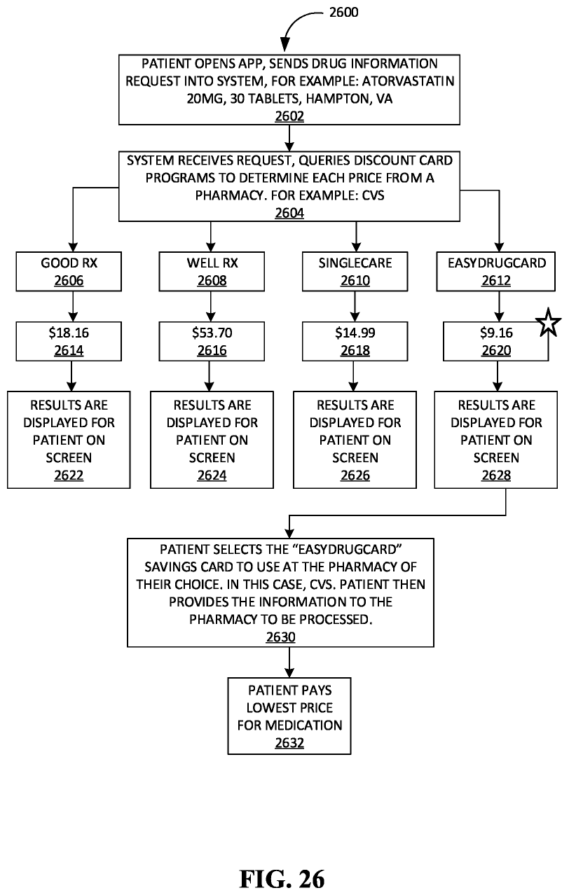

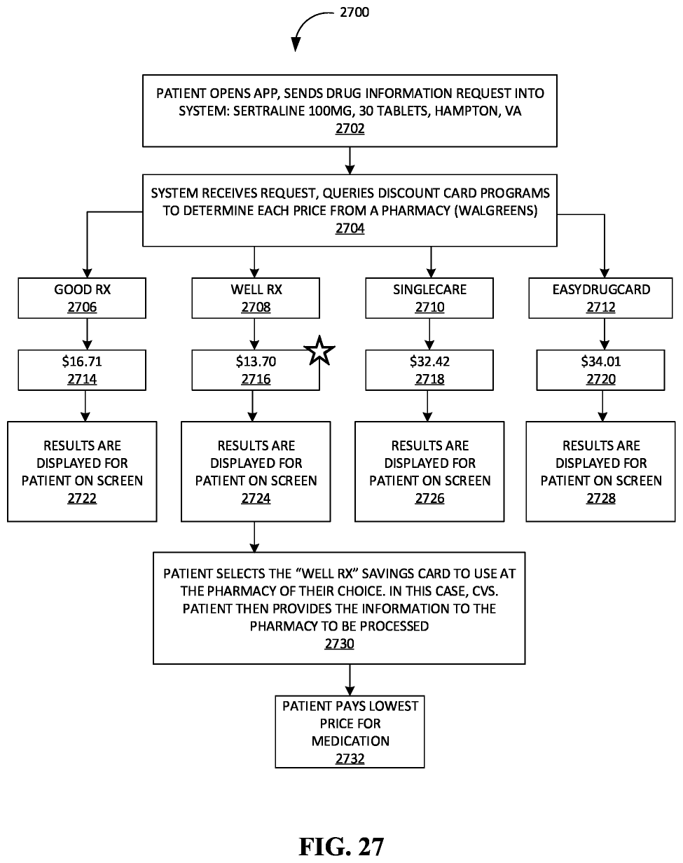

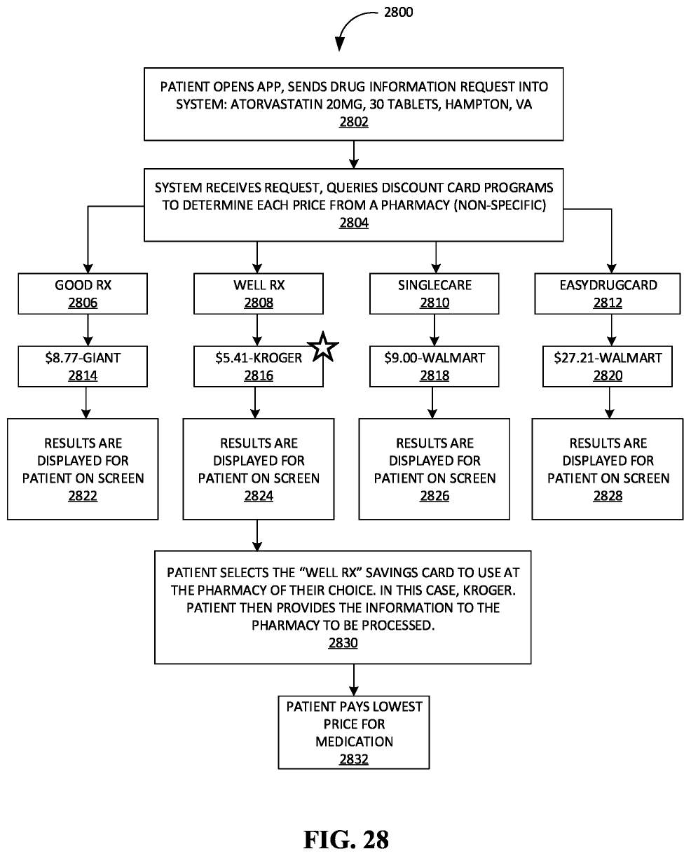

Further, the lowest price may be obtained based on a specific pharmacy, as shown in FIG. 26, representing CVS and Walgreens as shown in FIG. 27. FIGS. 28-31 illustrates the lowest prices are shown with no specified pharmacy, therefore it is the biggest discount that the card may obtain.

If the patient has multiple prescriptions, an additional feature is documentation within a software application that allows the patient to be able to keep up with which pharmacy consistently had the lowest prices for their medications, and recommend strictly based off on prices which pharmacy would be the best fit for a home pharmacy. This ultimately would empower the patient to continue achieving the highest discount while enabling the pharmacist at the home store to maintain a record of the person's prescriptions and preventing any possible drug-drug interactions that may arise. That is the only downfall of going from pharmacy to pharmacy, as the lack of prescription records inhibits the pharmacists from seeing if a person obtained a particular drug at another pharmacy, which in turn disallows them from detecting drug-drug interactions.

As seen from the diagram in FIG. 28, Kroger through WellRx had the lowest prices for a good amount of prescriptions. However, as prescriptions aren't the main focus/breadwinner as far as revenue goes, grocery stores indeed tend to have lower prices. One downfall is that there are more retail pharmacies than there are grocery stores. There are 2,782 Kroger stores nationally, vastly outnumbered compared to over 9,600 CVS (as of 2016) and 9,560 Walgreens (as of August 2018). In addition, if a medication is out of stock at a location, then the task begins of trying to find another store within a reasonable distance. Grocery store pharmacies also close early, often not open after 6 pm, while most pharmacies have extended hours, open 365 days a year and up to 24 hours a day. For these reasons and more, people tend to rely on retail pharmacies.

These functions would be performed by the AIS; and Automated Information System that has been programmed to perform these specified tasks as directed. An Automated Information System (AIS) is a system of computer hardware, computer software, data, and/or telecommunications that performs functions such as collecting, processing, storing, transmitting, and displaying information. For the disclosed software application, AIS may be a program that may process the incoming requests being made by consumers and patients in an attempt to search for the best medication price.

Further, the disclosed system provides a revolutionary tool to search for the most affordable prescription discount card. The disclosed system may be implemented on to the patient's smartphone device, tablet, and/or computer. The patient may be allowed access to not only compare medication prices between pharmacies like some prescription cards offer, but also between multiple discount cards themselves at the same time as well. Additionally, this cross-referencing from two databases using a simulation system puts the control of the drug price into the consumer's hands. The disclosed system aids consumers as increasing medication prices are affecting people across the globe. Further, the disclosed system may save the customer time and money, as well as ensure that they truly pay a competitive price for their medication. Further, the consumers (or patient) may be provided the location and shown directions with its built-in GPS. This may alleviate any confusion and energy the consumers have in the hunt for the most affordable prices possible. On almost every single discount card is a claim stating how much the card could save by using the card, without a method for discovering the true savings.

Further, the disclosed system may allow the patients to search and compare prices of medications between different drug cards at the same time. The disclosed system uses a computer program to send simulated requests to these discount card programs and bring back the price they would obtain on their site and comparing each of the findings against each other. The disclosed system may be configured for performing the shopping for the consumer, and consolidates the results in an easy-to-use mobile app. By using the same format and keeping the process simple, the disclosed system aims to simply while providing an increase in savings both in time and monetarily for the patient. This may be achieved by cross-referencing the data (prescription costs from the individual prescription cards) with the prices at different pharmacy locations to ensure the patient can ultimately find the lowest price for their medication within their location with ease simultaneously in a time sensitive manner

Further, the disclosed system may be configured for allowing the patient to be provided with directions to the pharmacy as well as options as to whether the patient may prefer a 24-hour pharmacy or not makes it that much more convenient to have access to the medications. In addition, if the patient generally prefers a single type of pharmacy, the patient may simply search for the desired pharmacy prices in that area, and likewise for any of the major pharmacy chains. The prices vary even within chains of pharmacies depending on the location. Further, some conventional technologies provide an average price to the patient, so the patient won't know the true price of their med until it is processed. The disclosed system doesn't base the prices off of one specific discount card, which is also different than the other applications, so that if a pharmacy isn't contracted with the card then it simply will show the other cards that are, and proceed to compare those listed on the formulary opposed to not being able to use that specific card at all. Further, the disclosed system may eliminate the time wasted looking for meds using other applications that won't apply.

Further, the disclosed software application may include a comparison application. More specifically, the comparison application serves as a means for patients to find and compare prices of medications between different drug cards in real-time. Further, the comparison application may be configured to any corresponding operating system. The comparison application may be associated with a client device, a database network, and an interface.

Further, the client device takes the form of any suitable device that can handle wireless information transfer and/or processing, working in conjunction with the database network and the interface. The client device may include but is not limited to smartphone devices, desktop computers, laptops, tablets, kiosks, and/or any other viable device.

Further, the database network may include any suitable server database network that may handle wireless information transfer, working in conjunction with the client device and the interface. The database network may include but is not limited to servers, local databases, and/or any other viable data transfer and management systems that support the client device and the interface. Further, the database network may be continuously updated to provide the user with up-to-date prescription discount data.

Further, the user may get a notification each month for their specific medication for the best price. Once a month, the comparison application may entail which drug card they recommend based on their customized “price range” after picking up the prescriptions for a few months. Further, there may be a consistently high and low range that the patient may see in an easy to interpret chart which tracks their spending on medications. Oftentimes, the patient has an idea of how much they spent but the disclosed system allows the patients to keep track of it for sure. This benefits the user, as they won't need to search monthly if they're getting the same medication.

Further, the disclosed system may be configured for providing an additional feature that may employ a “Prescription Card Wallet”. The prescription card wallet already includes image recognition technology so that it can scan a discount card (or multiple) and enter the information into the form to be used for a personalized comparison search. However, once scanned and used, the discount card may be saved into a “discount card wallet” for use at a later time when necessary. This may also be used to track which cards are used, the number of times, and the amount saved per prescription on average.

Further, the disclosed system may be configured for allowing the patients to choose the price they want to pay based on the search results from a prescription savings card, price, and pharmacy from which they wish to obtain your medication. Further, the disclosed system may help the cause amidst progress being made. Blink health for instance, as critically acclaimed as a money saver as it may be, isn't contracted with CVS or Walgreens, the two major retail pharmacy chains known today. So even if they did have the lowest price, the medication can't be filled at either pharmacy. At one point in time Blink Health was contracted with both, until each company dropped it off its formulary.

Year to year, pharmacy companies decide what they can do to maximize profit, some ways at the expense of the consumer even if means cutting off cards that provided extensive savings. Walgreens was first, then CVS followed suit later the same year. In one decision or contract signed, one drug savings card can be discarded for another in such a way that benefits both sides of the spectrum, leaving the consumer to figure out the logistics. In addition, during a quick search, Blink health doesn't have the lowest price for every medication. Blink charges a rate of $67.72 where you can “pay in the app” before going to the pharmacy so that no other discount cards can be applied afterward for comparison purposes. If they pay inside the app, even if a lower price is found a refund is unlikely. Further, companies play off the fact that prices rise and drop, which is indeed the case, but not as rapidly. Despite the claim, a similar search was performed for this same medication in the GoodRx app at Kroger, one of the pharmacies that Blink Health is contracted with, and the price is only $22.60. At a CVS, a pharmacy Blink is reportedly not contracted with, the price is still almost $4 less with GoodRx. In contrast, if a patient needed Atorvastatin, the price Blink health would provide is only $12.17 at Kroger, whereas at a CVS the price is more than double that, sitting at $27.32. The point is, neither discount card can guarantee you the lowest price, which requires the patient to have to shop around to determine which prescription application would be the best to use. No one savings card can offer the lowest price for every medication. Even other “comparison” websites claim to provide the lowest price but even they charge a little more in an attempt to make a profit off of the difference. Discount DetectoRx is an exemplary embodiment of the disclosed system herein, which may alleviate all of the extra work it takes to obtain the golden price.

Further, the disclosed system may allow the patients to search and compare prices of medications between different drug cards at the same time. It uses a computer program to send simulated requests to the discount card programs and display the price they would obtain on their site. The catch is that it then takes it a step further, by comparing each of these against each other. Further, the disclosed system performs the shopping for the consumer and consolidates the results in an easy-to-use mobile app. By using the same format and keeping the process just as simple, the disclosed system aims to simplify while providing an increase in savings both in time and monetarily for the patient. The may be achieved by cross-referencing the data (prescription costs from the individual prescription cards) with the prices at different pharmacy locations to ensure the patient may ultimately find the lowest price for their medication within their location with ease. These two main components combined with allowing the patient to be provided with directions to the pharmacy as well as options as to whether they prefer a 24-hour pharmacy or not make it that much more convenient to have to access the medications. In addition, if the patient generally prefers Walgreens or Rite Aid, the patient can simply filter the search for their prices in that area, and likewise for any of the major pharmacy chains. The prices vary even within chains of pharmacies depending on the location. On other apps an average price is provided to the patient, so the patient won't know the true price of their med until it is processed. Further, the disclosed system doesn't base the prices off of one specific discount card, which is also different than the other applications, so that if a pharmacy isn't contracted with the card then it simply will show the other cards that are, and proceed to compare those listed on the formulary opposed to not being able to use that specific card at all. This method may eliminate the time wasted looking for meds using other applications that won't apply. For instance, the price for Abilify (Aripiprazole) 5 mg, 30 tablets on blink health. The price came up to be $67.72, a coupon may not be found for this same drug on RetailMeNot Rx Saver, formerly known as LowestMed, another highly praised tool to lower prices on prescriptions. However, the medication may be found on GoodRx for a discounted price, at one specific pharmacy, Kroger. Depending on the drug, the best prescription drug card varies, because there isn't a means to showcase the lowest drug cost for every drug. There are many different methods from which individual drug cards companies obtain their price, but the disclosed system simply uses the prices that each provides to compare them. Unless there was a way to compare the drug prices from the actual cards themselves so the patients wouldn't have to spend extra time going from application to application in an attempt to find one that works the best. Further, this technology may be called DCS: The Drug Card Simulator. The goal of the drug card comparison application is to be the one-stop prescription drug mobile application that provides real-time prices and real options that allows the consumer to see which prescription drug card can and should be used. Further, the disclosed system may conserve even more time in the patient's process. Most, if not all of the prescription-saving applications will only allow the patient to look up one drug at a time. However, the disclosed system may allow the patient to search up to but not limited to 7 drugs at a time, and compare up to but not limited to 7 drug cards at a time, from which the lowest drug prices should be able to be obtained, all in under a minute. For example, if a patient came into the pharmacy with 7 drug cards they wanted to use, they would be able to type in all 7 drug cards, select the dosage forms, amount, formulary, and location, and see which of those present the lowest price. Tabs would appear on the side for those searching for multiple prescriptions that way if one savings card was able to secure a lower price for one drug as opposed to another then each would be easily accessible. Additional search tools such as pharmacy hours, reviews, and further drug information play a role as well, all to benefit the consumer. The patient may never overpay for a prescription again. Further, the disclosed system may be also available in but not limited to Spanish as well, at any time the consumer can switch between languages to suit their preference. Alternatively, if the consumer wishes not to want to type in all of their prescriptions and search for the dosage, etc. they can use the Prescription Card Recommender Tool. This utilizes the consumer data already presently gathered to determine which card may statistically be more beneficial by seeking which card on average saved the most money in the app on a specific medication instead of the consumer looking up the specific prices themselves.

Market demand: Consumers are paying out billions a year for prescriptions, and the patients are always looking for the next big thing to lower their drug prices, even more, to ensure they feel like they are getting the best deal. As of now, Americans pay about 40 cents to the dollar more than the next country for our prescriptions, the closest being Canada. By allowing them to physically see how they are not only comparing pharmacies but actual drug cards as well, the patients may feel confident and empowered when the patient may come to the pharmacy to pick up their prescription, leading to customer satisfaction, and hopefully ultimately greater patient compliance and improved health outcomes.

The disclosed application is catered to the patient's needs. If the patients are going to pay out of pocket, the patients may as well be paying the lowest price. In addition, if the patient wants to simply look at a list of all the top prescription discount cards themselves, there is an options tab where they will be listed, when the patient clicks on the picture linked to the one he/she wants to view, the application will send them to the webpage. This alleviates the patient from once again having to go online to Google™ or Bing™ and try to search for the best discount cards. The list of prescription discount cards in the database may be routinely updated to ensure the consumer is indeed receiving the best price. This list may be filtered by “most used” using our Prescription Savings Card Recommender Tool or simply “A-Z”.

Currently, in the pharmacy field, there are prescription drug cards and prescription drug card applications that present the consumer with one specific price through their system or program at multiple pharmacies. Further, the disclosed method utilizes an application that allows the patient to compare each of these drug card programs at the same time, which essentially minimizes the amount of time, effort, gas, and energy put towards researching the prices of prescriptions between different discount cards and pharmacies. Some conventional systems in place at the moment only allow a person to process the price of one prescription drug at one time which results in a lot of time spent from both the pharmacy and the patient having to type in the prescription details of the drug into each discount site individually if the patients are trying multiple cards to obtain the lowest price. This is a hassle for both sides of the window in a pharmacy. When a person brings multiple cards in with no idea of which would be most effective, it only slows down production and lengthens the lines, which end up in longer wait times, decreased customer satisfaction and most importantly more time patients are going without their medication that they already have to pay for monetarily. In addition, there is also a discrepancy because the price of medication differs at different locations. Even though the existing discount application may say that a certain price is for a certain chain of pharmacies, the actual price depends on the pharmacy store location itself, which frustrates both the consumer and the pharmacy technician/pharmacist at the time of billing. Therefore, in the event a patient is in an emergency or a severe medical crisis, often the situation leads to them having to settle for the first price they're presented at the pharmacy that's better than the cash price or listed under insurance, instead of obtaining the lowest price available. The employee assisting them may have only looked up one specific insurance card when if my application would've been used, the individual would've had access to all the savings in a fraction of the time. Instead, valuable time is lost in an attempt of the patient to find the lowest price, and the pharmacy employee having to process each drug card that the patient presents or may have on file.

Further, the disclosed system may minimize the effort needed to search for the lowest prescription drug card provided at the specific pharmacy location that the patient prefers.

Further, in an embodiment, the patient receives a prescription from the doctor's office, hospital, clinic, etc. Further, the patient downloads the comparison discount card application on their iPad, computer, or tablet, smartphone, or other computerized processing systems. Further, the patient opens up the application, types the name, dose, amount, and formulary of each of the drugs into its respective search bars. Further, if the patient wants to use the “Drug Card Recommender Tool”, the patients may select that option to reveal which prescription card statistically may be the best option, the lowest price isn't guaranteed. If the patient wishes to search the specific drug cards in hand, they may also type in the name of each savings card into the Drug Card Simulator to obtain the lowest price for each med out of each savings program that was provided. Further, the radius in miles of the location of a pharmacy where the patient prefers to pick up the medication can be adjusted, as well as filters/search tools such as the pharmacy hours, whether 24 hours or not, patient reviews, all listed from high to low, and on a rating system based on a scale of 0-5 stars. Further, the results of the patient view of the prices given by the discount cards can filter the results based on off-price and location, either within a certain mile radius or a specific city, state. The consumer then selects which prescription drug card meets their requirements. Further, the prescription card at the respective pharmacy that corresponds to the lowest price is selected. Further, the disclosed system may provide directions to the pharmacy, as well as its phone number and hours of operation to the pharmacy selected. Further, the patient may be directed to the drug prescription card page with the lowest price, and the patient will present the pharmacy with its specific card information to be processed. Further, the discount card is added to the prescription on the patient's profile in the pharmacy. Further, the patient pays for the most affordable prescription cost relevant to the customer's preferences each time. Further, when the specific medication is selected within the app, common brand names, as well as uses for the medication, will also be presented. Even though this tool may provide a specific discount for medications each visit, the prices may fluctuate, and so the patient may not pay the same amount each time, and the same card may not be the most beneficial each time either as it varies how the individual prescription savings cards obtain their discounts.

Further, the disclosed system may allow the customer to see which card provides the lowest price. In addition, the location search filter allows the patient to see the prices of the prescription discount card at different pharmacies within the radius selected so that patient can be confident in obtaining the lowest price at the specific location the patient may select. Oftentimes, some conventional applications may show the price of a prescription drug card at a pharmacy chain when the actual price can vary based on the physical location and demographic of the pharmacy. In the end, the patient becomes empowered through the lowest medication prices and the drug information to ensure they are indeed picking up the right medication as opposed to searching for the common uses on a search engine. That is yet another problem that consumers face. Once they obtain the prescription, especially if there are multiple, it can be difficult for them to keep up with the reason the patients have prescribed the medication.

Further, the disclosed system may be beneficial to everyone across the board. The patients and the pharmacies may find the disclosed system uses, as well as the discount companies who want their cards to at least be considered when patients are shopping for the lowest price. As of now, if the prescription card isn't one of the first results when a patient searches for a discount card, or in hand when a pharmacy technician/pharmacist is looking to use one to lower the price, it may most likely be overlooked. People without insurance may benefit the most using the disclosed system, but even people with insurance may find that their medications may be obtained at a low price than what their insurance plans are willing to cover.

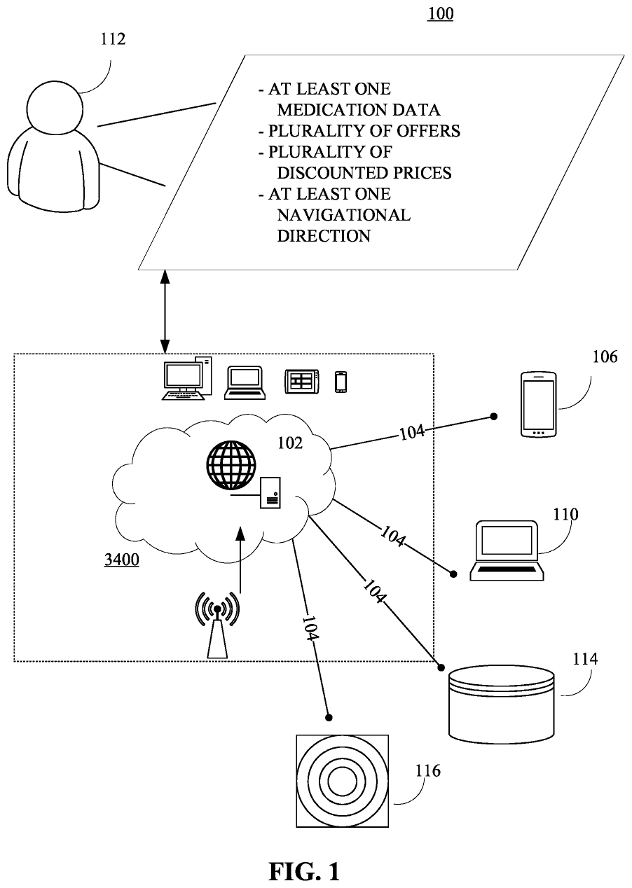

FIG. 1 is an illustration of an online platform 100 consistent with various embodiments of the present disclosure. By way of non-limiting example, the online platform 100 for facilitating managing purchase of medications may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to, the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 3400.

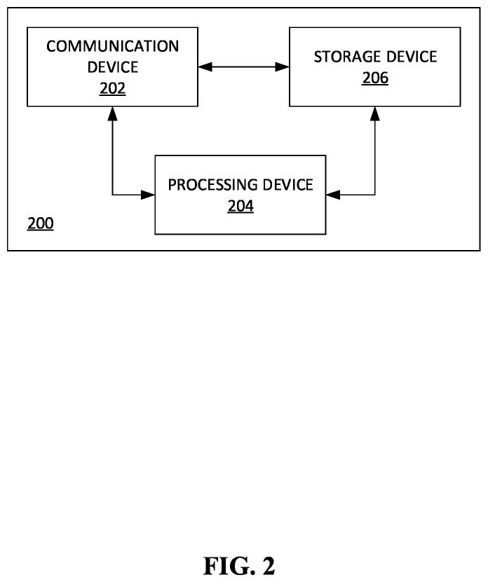

FIG. 2 is a block diagram of a system 200 for facilitating managing purchase of medications, in accordance with some embodiments. Accordingly, the system 200 may include a communication device 202, a processing device 204, and a storage device 206. Further, the communication device 202 may be configured for receiving at least one medication request data associated with a patient from at least one device. Further, the at least one medication request data may include at least one medication description of at least one medication and at least one patient identifier of the patient. Further, the communication device 202 may be configured for receiving a plurality of prices offered by a plurality of stores for the at least one medication from a plurality of store devices associated with the plurality of stores. Further, the communication device 202 may be configured for transmitting a plurality of discounted prices offered by the plurality of stores for the at least one medication, at least one discount card data, and a plurality of store data to the at least one device.

Further, the processing device 204 may be communicatively coupled with the communication device 202. Further, the processing device 204 may be configured for identifying the plurality of stores based on the at least one medication description. Further, the plurality of stores provides the at least one medication. Further, the processing device 204 may be configured for applying the at least one discount card data to the plurality of prices offered by the plurality of stores for the at least one medication. Further, the processing device 204 may be configured for generating the plurality of discounted prices offered by the plurality of stores for the at least one medication based on the applying.

Further, the storage device 206 may be communicatively coupled with the processing device 204. Further, the storage device 206 may be configured for retrieving the at least one discount card data of at least one discount card associated with the patient based on the at least one patient identifier. Further, the at least one discount card data may be used to reduce the plurality of prices offered by the plurality of stores for the at least one medication by at least one portion of the plurality of prices. Further, the storage device 206 may be configured for retrieving the plurality of store data associated with the plurality of stores based on the identifying.

Further, in some embodiments, the at least one medication request data may include at least one preference data associated with at least one preference of the patient. Further, the processing device 204 may be configured for analyzing the at least one preference data and the plurality of store data. Further, the processing device 204 may be configured for selecting at least one store from the plurality of stores based on the analyzing. Further, the communication device 202 may be configured for transmitting at least one discounted price offered by the at least one store for the at least one medication and at least one store data of the at least one store to the at least one device.

Further, in some embodiments, the at least one preference data may include at least one location preference data associated with at least one location preference of the at least one preference. Further, the plurality of store data may include a plurality of store location data associated with a plurality of store locations of the plurality of stores. Further, the analyzing may include analyzing the at least one location preference data and the plurality of store location data. Further, the selecting of the at least one store may be further based on the analyzing of the at least one location preference data and the plurality of store location data.

Further, in some embodiments, the at least one device may include a location sensor. Further, the location sensor may be configured for generating patient location data based on a location of the patient. Further, the at least one location preference data may include the patient location data.

Further, in some embodiments, the processing device 204 may be configured for analyzing the patient location data and at least one store location data associated with the at least one store based on the selecting. Further, the processing device 204 may be configured for generating at least one navigational direction from the location of the patient to at least one store location of the at least one store. Further, the communication device 202 may be configured for transmitting the at least one navigational direction to the at least one device.

Further, in some embodiments, the at least one preference data may include at least one purchase time preference data associated with at least one purchase time preference of the at least one preference. Further, the plurality of store data may include a plurality of store operating duration data associated with a plurality of store operating durations of the plurality of stores. Further, the analyzing may include analyzing the at least one purchase time preference data and the plurality of store operating duration data. Further, the selecting of the at least one store may be based on the analyzing of the at least one purchase time preference data and the plurality of store operating duration data.

Further, in some embodiments, the processing device 204 may be configured for analyzing the plurality of discounted prices offered by the plurality of stores for the at least one medication. Further, the processing device 204 may be configured for determining a lowest discounted price from the plurality of discounted prices based on the analyzing of the plurality of discounted prices. Further, the processing device 204 may be configured for identifying a store offering the lowest discounted price from the plurality of stores based on the determining of the lowest discounted price. Further, the communication device 202 may be configured for transmitting the lowest discounted price offered by the store for the at least one medication and store data of the store to the at least one device.

Further, in some embodiments, the communication device 202 may be configured for receiving the at least one discount card data from the at least one device. Further, the at least one device may include at least one scanning device. Further, the at least one scanning device may be configured for generating the at least one discount card data based on scanning at least one visual representation associated with the at least one discount card. Further, the storage device 206 may be configured for storing the at least one discount card data.

Further, in some embodiments, the at least one discount card data may include at least one redeeming restriction associated with a redeeming of the at least one discount card for the at least one medication at the plurality of stores. Further, the processing device 204 may be configured for analyzing the at least one redeeming restriction with at least one of the at least one medication description and the plurality of store data using at least one machine learning model. Further, the processing device 204 may be configured for identifying at least one redeemable discount card of the at least one discount card, a redeemable medication of the at least one medication, and at least one redeemable store of the plurality of stores based on the analyzing of the at least one redeeming restriction. Further, the at least one redeemable discount card may be redeemable for the redeemable medication at the at least one redeemable store. Further, the processing device 204 may be configured for applying at least one redeemable discount card data of the at least one redeemable discount card to at least one price offered by the at least one redeemable store for the redeemable medication. Further, the processing device 204 may be configured for determining at least one discounted price offered by the at least one redeemable store for the redeemable medication based on the applying of the at least one redeemable discount card. Further, the communication device 202 may be configured for transmitting the at least one discounted price offered by the at least one redeemable store for the redeemable medication to the at least one device.

Further, in some embodiments, the communication device 202 may be configured for receiving at least one store selection from the at least one device. Further, the communication device 202 may be configured for receiving at least one discount card selection from the at least one device. Further, the communication device 202 may be configured for transmitting an order reference to the at least one device. Further, the processing device 204 may be configured for identifying at least one store from the plurality of stores based on the at least one store selection. Further, the processing device 204 may be configured for identifying at least one selected discount card from the at least one discount card based on the at least one discount card selection. Further, the processing device 204 may be configured for applying at least one selected discount card data of the at least one selected discount card to at least one price offered by the at least one store for the at least one medication. Further, the processing device 204 may be configured for determining at least one discounted price offered by the at least one store for the at least one medication based on the applying of the at least one first discount card. Further, the processing device 204 may be configured for generating the order reference for the purchase of the at least one medication from the at least one store based on the determining of the at least one discounted price.

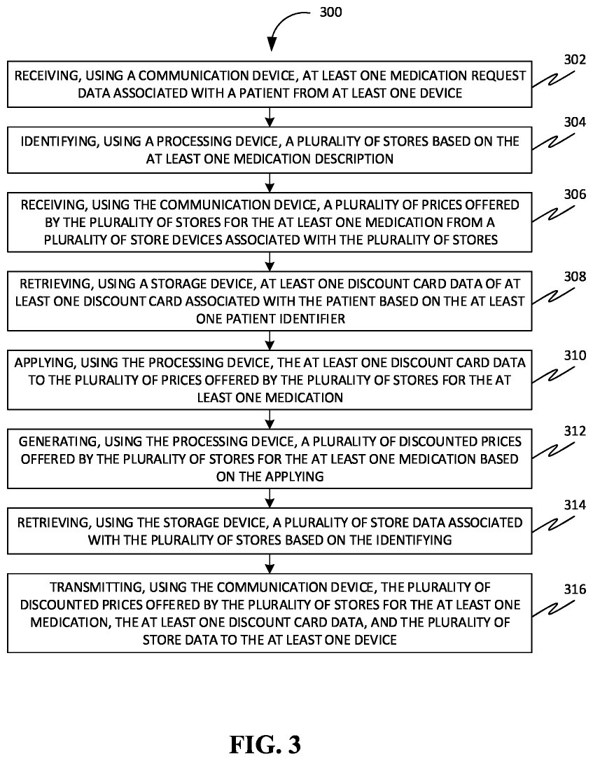

FIG. 3 is a flowchart of a method 300 for facilitating managing purchase of medications, in accordance with some embodiments. Accordingly, at 302, the method 300 may include receiving, using a communication device (such as the communication device 202), at least one medication request data associated with a patient from at least one device. Further, the at least one medication request data may include at least one medication description of at least one medication and at least one patient identifier of the patient.

Further, at 304, the method 300 may include identifying, using a processing device (such as the processing device 204), a plurality of stores based on the at least one medication description. Further, the plurality of stores provides the at least one medication.

Further, at 306, the method 300 may include receiving, using the communication device, a plurality of prices offered by the plurality of stores for the at least one medication from a plurality of store devices associated with the plurality of stores.

Further, at 308, the method 300 may include retrieving, using a storage device (such as the storage device 206), at least one discount card data of at least one discount card associated with the patient based on the at least one patient identifier. Further, the at least one discount card data may be used to reduce the plurality of prices offered by the plurality of stores for the at least one medication by at least one portion of the plurality of prices.

Further, at 310, the method 300 may include applying, using the processing device, the at least one discount card data to the plurality of prices offered by the plurality of stores for the at least one medication.

Further, at 312, the method 300 may include generating, using the processing device, a plurality of discounted prices offered by the plurality of stores for the at least one medication based on the applying.

Further, at 314, the method 300 may include retrieving, using the storage device, a plurality of store data associated with the plurality of stores based on the identifying.

Further, at 316, the method 300 may include transmitting, using the communication device, the plurality of discounted prices offered by the plurality of stores for the at least one medication, the at least one discount card data, and the plurality of store data to the at least one device.

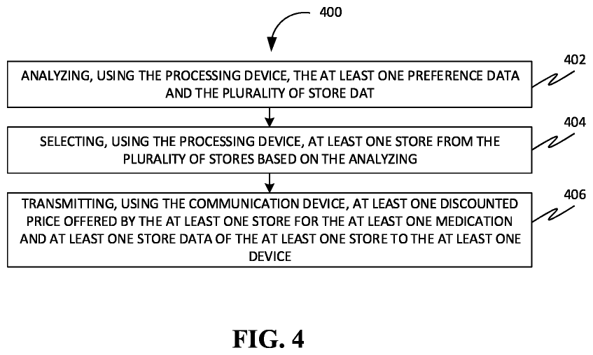

FIG. 4 is a flowchart of a method 400 for selecting at least one store for facilitating managing the purchase of medications, in accordance with some embodiments.

Accordingly, the at least one medication request data may include at least one preference data associated with at least one preference of the patient. Further, at 402, the method 400 may include analyzing, using the processing device, the at least one preference data and the plurality of store data.

Further, at 404, the method 400 may include selecting, using the processing device, at least one store from the plurality of stores based on the analyzing.

Further, at 406, the method 400 may include transmitting, using the communication device, at least one discounted price offered by the at least one store for the at least one medication and at least one store data of the at least one store to the at least one device.

Further, in some embodiments, the at least one preference data may include at least one location preference data associated with at least one location preference of the at least one preference. Further, the plurality of store data may include a plurality of store location data associated with a plurality of store locations of the plurality of stores. Further, the analyzing may include analyzing the at least one location preference data and the plurality of store location data. Further, the selecting of the at least one store may be further based on the analyzing of the at least one location preference data and the plurality of store location data.

Further, in some embodiments, the at least one device may include a location sensor. Further, the location sensor may be configured for generating patient location data based on a location of the patient. Further, the at least one location preference data may include the patient location data.

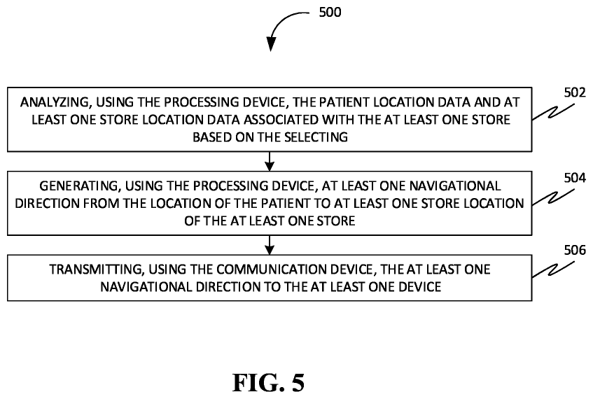

FIG. 5 is a flowchart of a method 500 for generating at least one navigational direction for facilitating managing the purchase of medications, in accordance with some embodiments.

Further, at 502, the method 500 may include analyzing, using the processing device, the patient location data and at least one store location data associated with the at least one store based on the selecting.

Further, at 504, the method 500 may include generating, using the processing device, at least one navigational direction from the location of the patient to at least one store location of the at least one store.

Further, at 506, the method 500 may include transmitting, using the communication device, the at least one navigational direction to the at least one device.

Further, in some embodiments, the at least one preference data may include at least one purchase time preference data associated with at least one purchase time preference of the at least one preference. Further, the plurality of store data may include a plurality of store operating duration data associated with a plurality of store operating durations of the plurality of stores. Further, the analyzing may include analyzing the at least one purchase time preference data and the plurality of store operating duration data. Further, the selecting of the at least one store may be based on the analyzing of the at least one purchase time preference data and the plurality of store operating duration data.

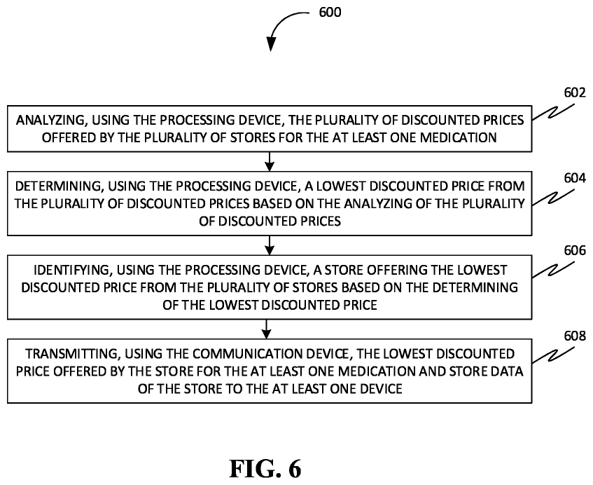

FIG. 6 is a flowchart of a method 600 for identifying a store offering the lowest discounted price for facilitating managing the purchase of medications, in accordance with some embodiments. Further, at 602, the method 600 may include analyzing, using the processing device, the plurality of discounted prices offered by the plurality of stores for the at least one medication.

Further, at 604, the method 600 may include determining, using the processing device, a lowest discounted price from the plurality of discounted prices based on the analyzing of the plurality of discounted prices.

Further, at 606, the method 600 may include identifying, using the processing device, a store offering the lowest discounted price from the plurality of stores based on the determining of the lowest discounted price.

Further, at 608, the method 600 may include transmitting, using the communication device, the lowest discounted price offered by the store for the at least one medication and store data of the store to the at least one device.

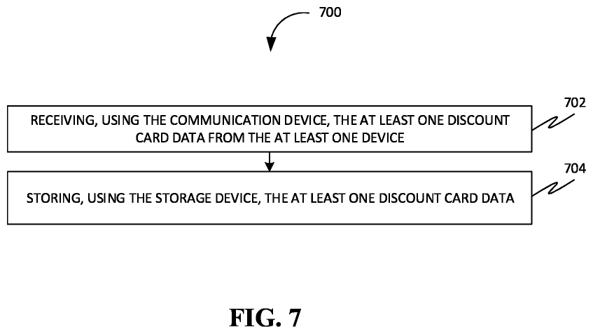

FIG. 7 is a flowchart of a method 700 for receiving the at least one discount card data for facilitating managing the purchase of medications, in accordance with some embodiments. Further, at 702, the method 700 may include receiving, using the communication device, the at least one discount card data from the at least one device. Further, the at least one device may include at least one scanning device. Further, the at least one scanning device may be configured for generating the at least one discount card data based on scanning at least one visual representation associated with the at least one discount card.

Further, at 704, the method 700 may include storing, using the storage device, the at least one discount card data.

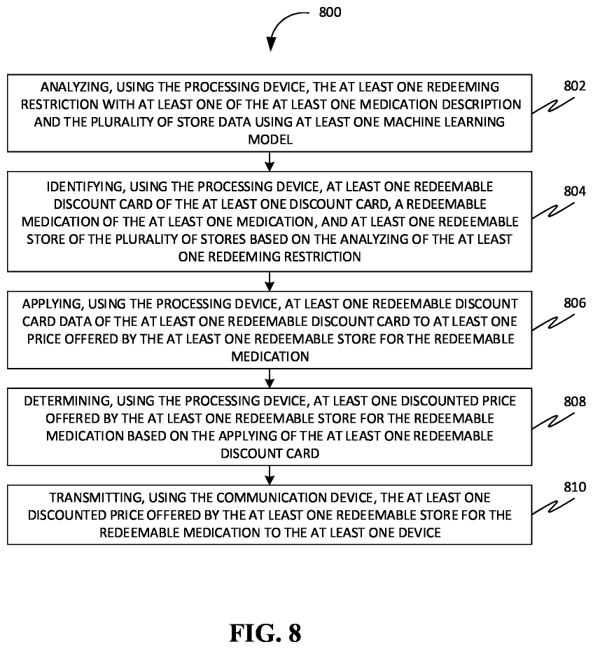

FIG. 8 is a flowchart of a method 800 for determining at least one discounted price for facilitating managing the purchase of medications, in accordance with some embodiments. Accordingly, the at least one discount card data may include at least one redeeming restriction associated with a redeeming of the at least one discount card for the at least one medication at the plurality of stores. Further, at 802, the method 800 may include analyzing, using the processing device, the at least one redeeming restriction with at least one of the at least one medication description and the plurality of store data using at least one machine learning model.

Further, at 804, the method 800 may include identifying, using the processing device, at least one redeemable discount card of the at least one discount card, a redeemable medication of the at least one medication, and at least one redeemable store of the plurality of stores based on the analyzing of the at least one redeeming restriction. Further, the at least one redeemable discount card may be redeemable for the redeemable medication at the at least one redeemable store.

Further, at 806, the method 800 may include applying, using the processing device, at least one redeemable discount card data of the at least one redeemable discount card to at least one price offered by the at least one redeemable store for the redeemable medication.

Further, at 808, the method 800 may include determining, using the processing device, at least one discounted price offered by the at least one redeemable store for the redeemable medication based on the applying of the at least one redeemable discount card.

Further, at 810, the method 800 may include transmitting, using the communication device, the at least one discounted price offered by the at least one redeemable store for the redeemable medication to the at least one device.

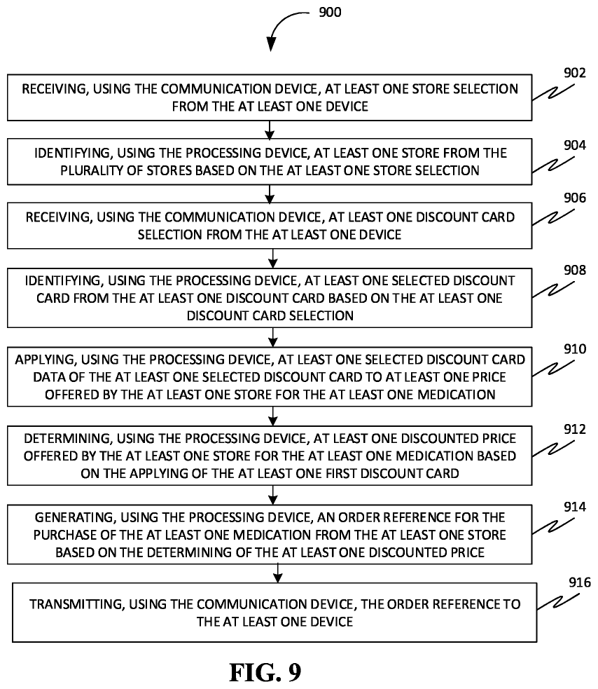

FIG. 9 is a flowchart of a method 900 for generating an order reference for facilitating managing the purchase of medications, in accordance with some embodiments. Further, at 902, the method 900 may include receiving, using the communication device, at least one store selection from the at least one device.

Further, at 904, the method 900 may include identifying, using the processing device, at least one store from the plurality of stores based on the at least one store selection.

Further, at 906, the method 900 may include receiving, using the communication device, at least one discount card selection from the at least one device.

Further, at 908, the method 900 may include identifying, using the processing device, at least one selected discount card from the at least one discount card based on the at least one discount card selection.

Further, at 910, the method 900 may include applying, using the processing device, at least one selected discount card data of the at least one selected discount card to at least one price offered by the at least one store for the at least one medication.

Further, at 912, the method 900 may include determining, using the processing device, at least one discounted price offered by the at least one store for the at least one medication based on the applying of the at least one first discount card.

Further, at 914, the method 900 may include generating, using the processing device, an order reference for the purchase of the at least one medication from the at least one store based on the determining of the at least one discounted price.

Further, at 916, the method 900 may include transmitting, using the communication device, the order reference to the at least one device.

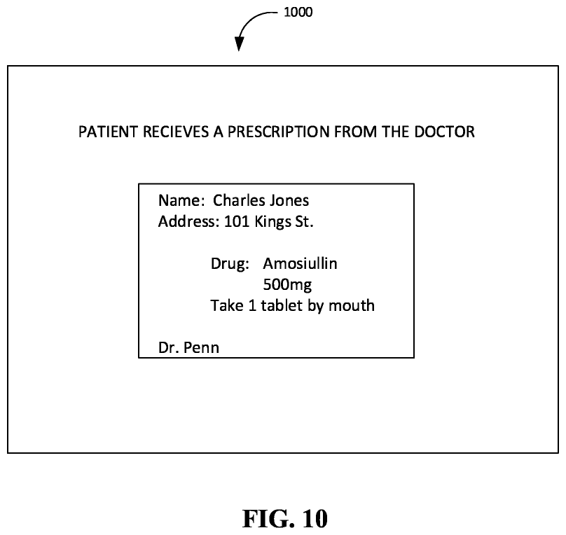

Further, as shown in FIGS. 10-25, the interface takes the form of the software platform of the comparison application. More specifically, the interface serves as the user-input communication platform, working in conjunction with the client device and the database network in providing the user with the means to find and compare prices of medications between different drug cards in real time, dependent on user-input credentials and parameters. In the preferred embodiment of the present invention, user-input credentials can be dictated by a received prescription from a doctor, as shown in FIG. 10. The interface may include a downloader, a launcher, a first search page, a second search page, a third search, an adder prompt, a scanner prompt, a buffer, a filter, a second list, and a window.

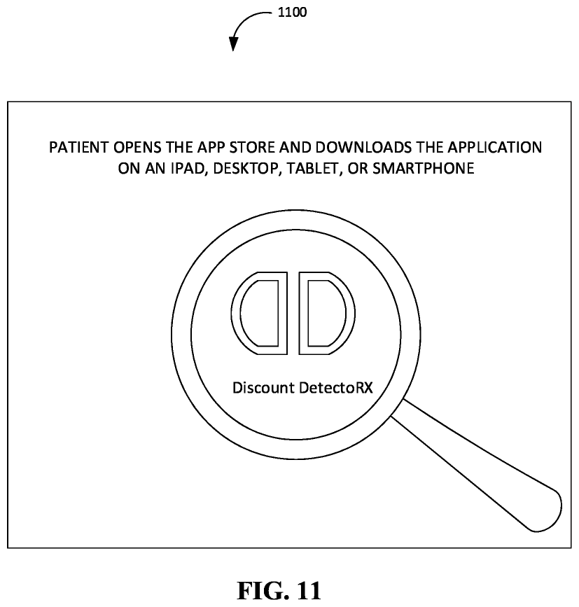

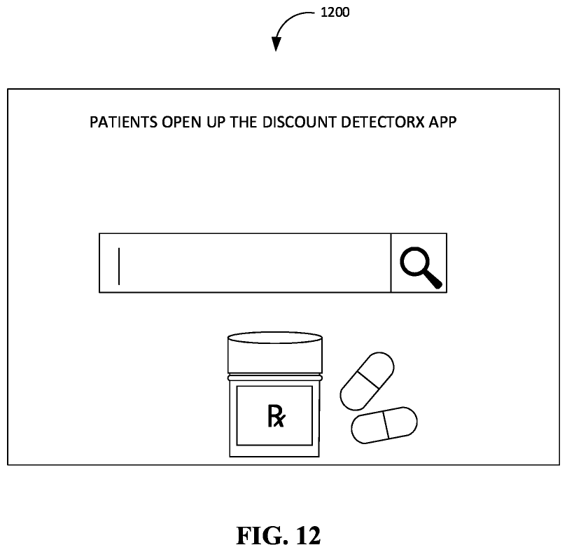

Further, as shown in FIG. 11, the downloader is incorporated in the comparison application. More specifically, the downloader allows the user to download the interface on the client device. Further, as shown in FIG. 12, the launcher proceeds the interface. More specifically, the launcher allows the user to start the interface.

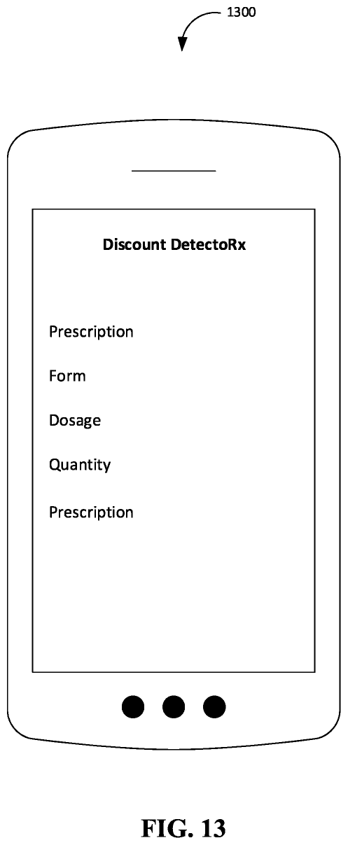

Further, as shown in FIG. 13, the first search page may be introduced upon the launcher start. More specifically, the first search allows the user to input medication credentials associated with the doctor's prescription shown in FIG. 10. Further, the first search page works in conjunction with the database network, pulling data from the associative medication database. The first search may include a plurality of queries.

Further, as shown in FIG. 13, the plurality of queries may be incorporated in the first search. More specifically, the plurality of queries may include fillable relevant fields that allow the user to input medication credentials associated with the doctor's prescription shown in FIG. 10. The medication credentials may include but are not limited to medication quantity, dosage, formula, etc.

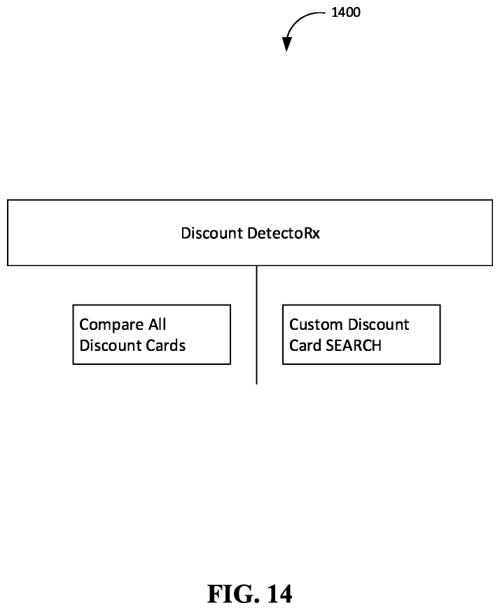

Further, as shown in FIG. 14, the second search page proceeds the first search page, once the plurality of queries has been fulfilled. More specifically, the second search page allows the user to search prescription savings. Further, a second search associated with the second search page may statistically compare all discount cards available in the database network, and/or allows the user to initiate a custom discount card search. The second search page comprises a first list, an inquiry tab, and an organizer.

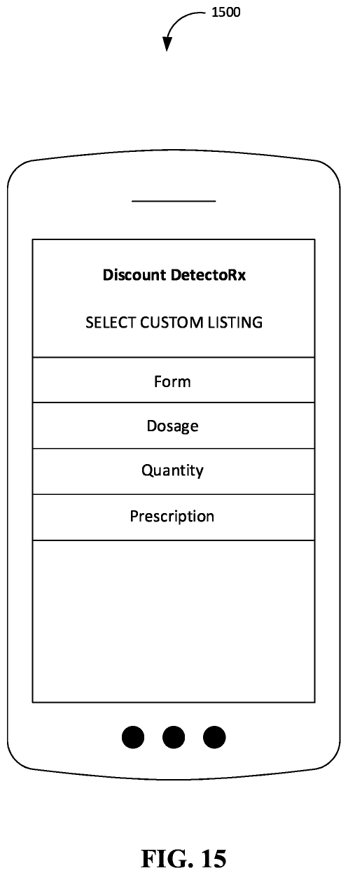

Further, as shown in FIG. 15, a first list is incorporated within the second search page. The first list displays a list of all the discount cards pulled from the database network in the second search. In reference to FIG. 15, an inquiry tab resides within the second search. More specifically, the inquiry tab allows the user to search specific discount card results from the second search. In reference to FIG. 15, an organizer is incorporated within the second search. More specifically, the organizer allows the user to arrange the listed discount cards. Further, the organizer may arrange the listed discount cards through alphabetical order, most frequently used, most recommended, highest percentage savings yield, and/or any other arrangement means.

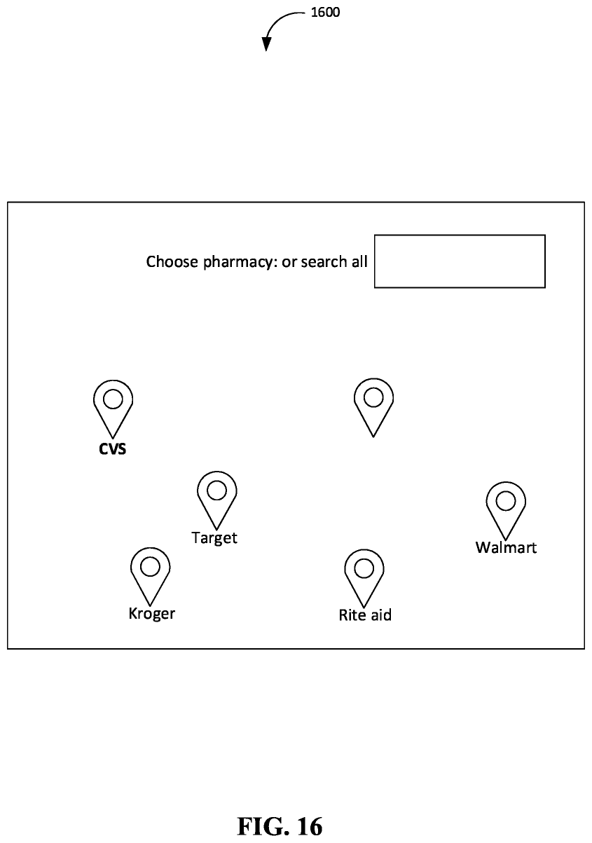

Further, as shown in FIG. 16, the third search may be incorporated within the interface. More specifically, the third search may include a GPS-based search engine that displays supporting pharmacies in the area. Further, the user may set the location radius where the user is willing to travel to pick up the medication. Further, the third search may also be arranged in a listed format, such that the user can search all supporting pharmacies in the area. The listed pharmacy results can be selected to display hours of operation, address, and contact information. The third search may include a map.

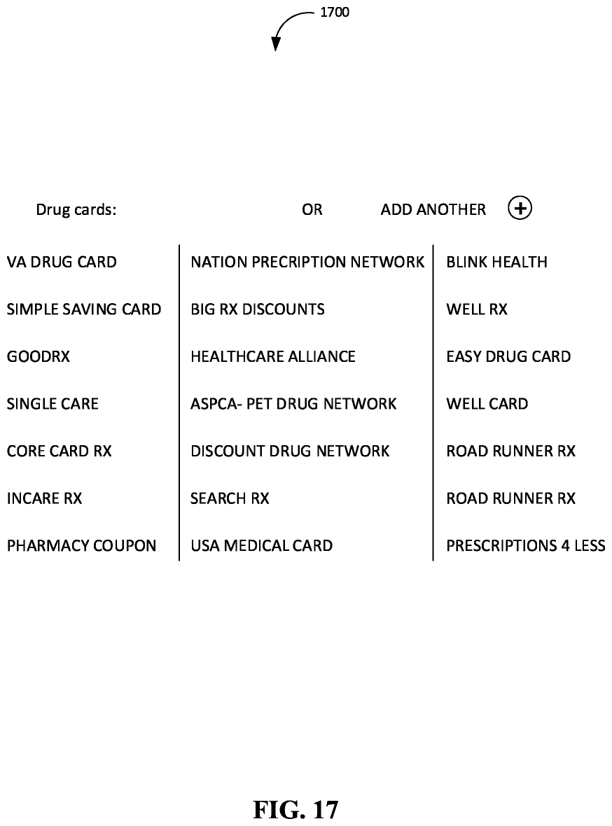

Further, as shown in FIG. 16, the map may be incorporated within the third search page. More specifically, the third search displays a topological representation of the searched pharmacies within an area. In reference to FIG. 17, the adder prompt may be incorporated within the interface. More specifically, the adder prompt allows users to add their own discount card if their card is not listed in the second search.

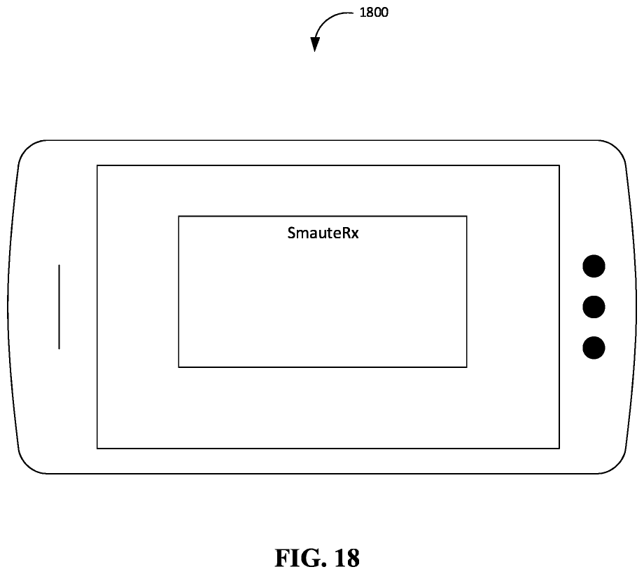

Further, as shown in FIG. 18, the scanner prompt may be incorporated within the interface. More specifically, the scanner prompt utilizes the client device scanner/camera hardware to facilitate quick-read (QR) code means of inputting data within the interface.

Further, the scanner prompt may scan QR codes through a corresponding QR code found on prescription discount cards, allowing the interface to automatically upload the discount card from the database network, and/or automatically fills in the required parameters associated with the adder prompt. The scanner prompt may include a type input.

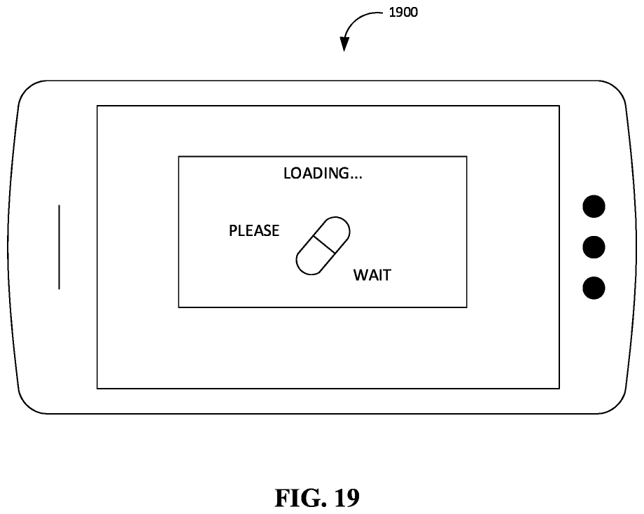

Further, as shown in FIG. 18, the type input allows the user to manually input a QR text code to automatically pull up/fill the discount card desired. In reference to FIG. 19, the buffer is incorporated within the interface. More specifically, the buffer works in conjunction with the scanner prompt, authenticating the QR input. The buffer comprises a verifier.

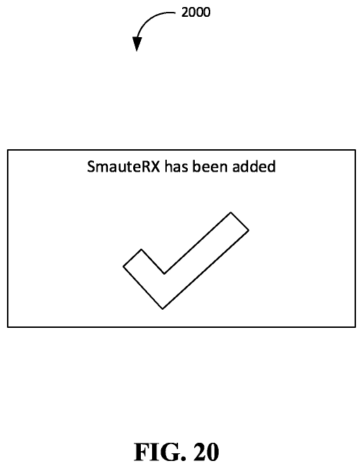

In reference to FIG. 20, the verifier may be incorporated within the buffer. More specifically, the verifier confirms the QR input has successfully been recognized by the interface, pulling up the associative information from the database network.

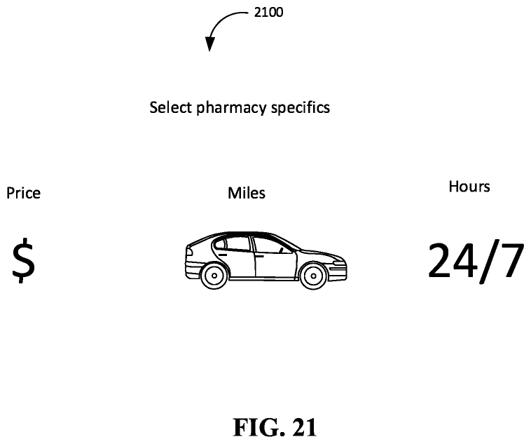

In reference to FIG. 21, the filter may be incorporated within the interface. More specifically, the filter allows the user to input pharmacy-specific parameters, working in conjunction with the third search, narrowing down the results listed from the third search. The filter comprises a plurality of conditions.

In reference to FIG. 21, the plurality of conditions may be incorporated within the filter prompt. More specifically, the plurality of conditions may include selectable pharmacy-specific parameters that narrow down the search results of the third search to the corresponding parameters. The pharmacy-specific parameters may include but are not limited to pharmacy price, distance, opening hours, etc.

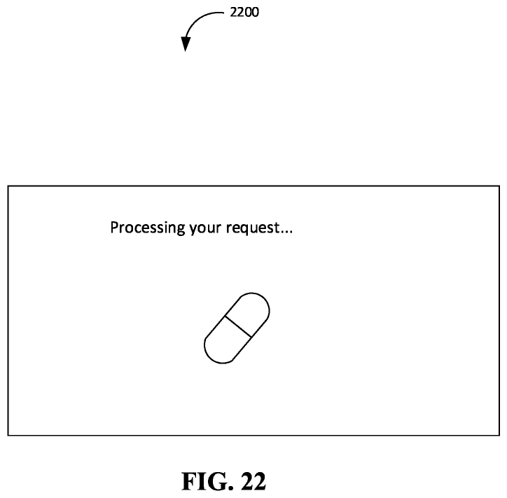

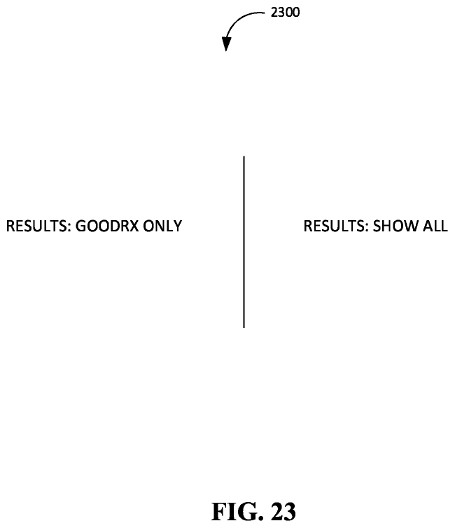

Further, as shown in FIGS. 22-23, the second list may be incorporated within the interface. More specifically, the second list displays the prescription prices based on the first search, the second search, and the third search input parameters. The second list may include a plurality of offers, a first tab, and a second tab.

Further, as shown in FIGS. 22-23, the plurality of offers may be incorporated within the second list. More specifically, the plurality of offers may include selectable prescription price results based on the input parameters of the first search, the second search, and the third search. In reference to FIG. 23, the first tab may be incorporated within the second list. More specifically, the plurality of offers allows the user to choose to see all prices through a selected prescription card input dictated by the second search.

Further, as shown in FIG. 23, the second tab may be incorporated within the second list. More specifically, the second tab allows the user to choose to see all prices of all corresponding prescription card inputs dictated by the second search.

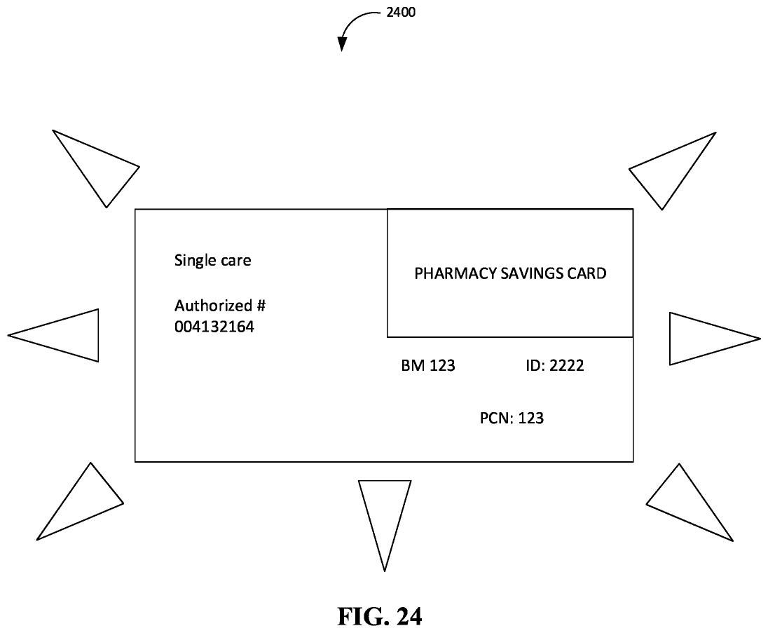

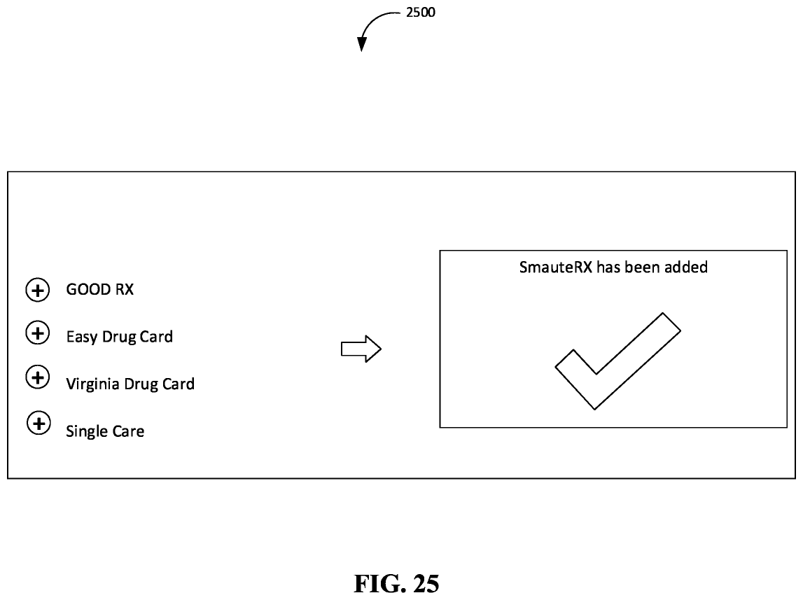

Further, as shown in FIGS. 24-25, the window page proceeds the second list upon the selection of an offer displayed on the second list. More specifically, the window allows the user to finalize and confirm their prescription order dictated by the first search, the second search, and the third search. further, the fulfillment of the window page outputs an order reference. The order reference may provide a sufficient report of the discount applied to the desired prescription with the selected prescription card. In one instance, the report generated may be printed, and/or handled digitally through the corresponding pharmacy. The window may include a plurality of first choices and a plurality of second choices.

Further, as shown in FIG. 24, the plurality of first choices resides in the window page. More specifically, the plurality of first choices allows the user to select the desirable medication at the price they find most desirable, dictated by the first search, the second search, and the third search parameters. Further, the desired medication selection may be prioritized to display the most affordable prescription costs.

Further, as shown in FIG. 24, the plurality of second choices resides in the window page. More specifically, the plurality of second choices allows the user to select a desired discount card, dictated by the first search, the second search, and the third search parameters. Further, the desired discount card may be prioritized to display the discount cards the yield the highest discount value applied to the plurality of first choices.

FIG. 10 is a screenshot of a user interface 1000 associated with a software application for facilitating managing purchase of medications, in accordance with some embodiments.

FIG. 11 is a screenshot of a user interface 1100 associated with the software application, in accordance with some embodiments.

FIG. 12 is a screenshot of a user interface 1200 associated with the software application, in accordance with some embodiments.

FIG. 13 is a screenshot of a user interface 1300 associated with the software application, in accordance with some embodiments. Further, the user interface 1300 may allow entering of the patient types in medication and searches database, selecting the correct quantity, dose, and formula.

FIG. 14 is a screenshot of a user interface 1400 associated with the software application, in accordance with some embodiments. Further, the patient may search for their prescription saving using a custom discount card search using the user interface 1400.

FIG. 15 is a screenshot of a user interface 1500 associated with the software application, in accordance with some embodiments. Further, the user interface 1500 may present a list of all discount cards in a general search. Further, the list may be listed by a-z, most frequently used, most recommended, and highest % savings yield.

FIG. 16 is a screenshot of a user interface 1600 associated with the software application, in accordance with some embodiments.

FIG. 17 is a screenshot of a user interface 1700 associated with the software application, in accordance with some embodiments. Further, the user interface 1700 may allow the patient to add their discount card if their card is not listed on the general search.

FIG. 18 is a screenshot of a user interface 1800 associated with the software application, in accordance with some embodiments. Further, the user interface 1800 may allow a user of a built in scanner in the at least one device to obtain or simply type the discount card in the at least one device.

FIG. 19 is a screenshot of a user interface 1900 associated with the software application, in accordance with some embodiments.

FIG. 20 is a screenshot of a user interface 2000 associated with the software application, in accordance with some embodiments.

FIG. 21 is a screenshot of a user interface 2100 associated with the software application, in accordance with some embodiments. Further, the user interface 2100 may allow the patient to select pharmacy specifics.

FIG. 22 is a screenshot of a user interface 2200 associated with the software application, in accordance with some embodiments. Further, the user interface 2200 may present prices with the pharmacy and prescription cards.

FIG. 23 is a screenshot of a user interface 2300 associated with the software application, in accordance with some embodiments. Further, the user interface 2300 may allow the patient to choose to see all prices through one business card or to view all from a list.

FIG. 24 is a screenshot of a user interface 2400 associated with the software application, in accordance with some embodiments. Further, the user interface 2400 displays the patient selection of the needed meds at the price the patient finds most desirable.

FIG. 25 is a screenshot of a user interface 2500 associated with the software application, in accordance with some embodiments. Further, the user interface 2500 may allow the patient to select the discount care of their choice.

FIG. 26 is a flow diagram of a method 2600 for facilitating comparing prices of medications between different drug discount cards, in accordance with some embodiments. Accordingly, at 2602, the method 2600 may include a patient opening the software application and sending drug information request for medication. Further, at 2604, the method 2600 may include a system (such as the system 200) receiving requests and queries. Further, the discount cards may include good RX 2606, Well RX 2608, single care 2610, and EasyDrug card 2612. Further, the good RX 2606 may be associated with a price 2614 of $18.16. Further, the Well RX 2608 may be associated with a price 2616 of $53.70. Further, the singlecare 2610 may be associated with a price 2618 of $14.99. Further, the easydrug card 2612 may be associated with a price 2620 of $9.16. Further, at 2622, the method 2600 may include displaying the price 2614 for the patient on a screen. Further, at 2624, the method 2600 may include displaying the price 2616 for the patient on the screen. Further, at 2626, the method 2600 may include displaying the price 2618 for the patient on the screen. Further, at 2628, the method 2600 may include displaying the price 2620 for the patient on the screen. Further, at 2630, the method 2600 may include the patient selecting the EasyDrug card 2612 to use at a pharmacy of their choice, in an instance, the pharmacy may be CVS. Further, at 2632, the method 2600 may include paying a lowest price for the medication.

FIG. 27 is a flow diagram of a method 2700 for facilitating comparing prices of medications between different drug discount cards, in accordance with some embodiments. Accordingly, at 2702, the method 2700 may include a patient opening the software application and sending drug information request for medication. Further, at 2704, the method 2700 may include a system (such as the system 200) receiving requests and queries discount card programs. Further, the discount cards may include good RX 2706, Well RX 2708, single care 2710, and EasyDrug card 2712. Further, the good RX 2706 may be associated with a price 2714 of $16.71. Further, the Well RX 2708 may be associated with a price 2716 of $13.70. Further, the singlecare 2710 may be associated with a price 2718 of $32.42. Further, the EasyDrug card 2712 may be associated with a price 2720 of $34.01. Further, at 2722, the method 2700 may include displaying the price 2714 for the patient on a screen. Further, at 2724, the method 2700 may include displaying the price 2716 for the patient on the screen. Further, at 2726, the method 2700 may include displaying the price 2718 for the patient on the screen. Further, at 2728, the method 2700 may include displaying the price 2720 for the patient on the screen. Further, at 2730, the method 2700 may include the patient selecting the Well RX card 2708 to use at a pharmacy of their choice, in an instance, the pharmacy may be CVS. Further, at 2732, the method 2700 may include paying a lowest price for the medication.

FIG. 28 is a flow diagram of a method 2800 for facilitating comparing prices of medications between different drug discount cards, in accordance with some embodiments. Accordingly, at 2802, the method 2800 may include a patient opening the software application and sending drug information request for medication. Further, at 2804, the method 2800 may include a system (such as the system 200) receiving requests and queries discount card programs. Further, the discount cards may include good RX 2806, Well RX 2808, singlecare 2810, and EasyDrug card 2812. Further, the good RX 2806 may be associated with a price 2814 of $8.77 at Giant pharmacy. Further, the Well RX 2808 may be associated with a price 2816 of $5.41 at Kroger. Further, the singlecare 2810 may be associated with a price 2818 of $9.00 at Walmart. Further, the EasyDrug card 2812 may be associated with a price 2820 of $27.21 at Walmart. Further, at 2822, the method 2800 may include displaying the price 2814 for the patient on a screen. Further, at 2824, the method 2800 may include displaying the price 2816 for the patient on the screen. Further, at 2826, the method 2800 may include displaying the price 2818 for the patient on the screen. Further, at 2828, the method 2800 may include displaying the price 2820 for the patient on the screen. Further, at 2830, the method 2800 may include the patient selecting the Well RX card 2808 to use at a pharmacy of their choice, in an instance, the pharmacy may be Kroger. Further, at 2832, the method 2800 may include paying a lowest price for the medication.

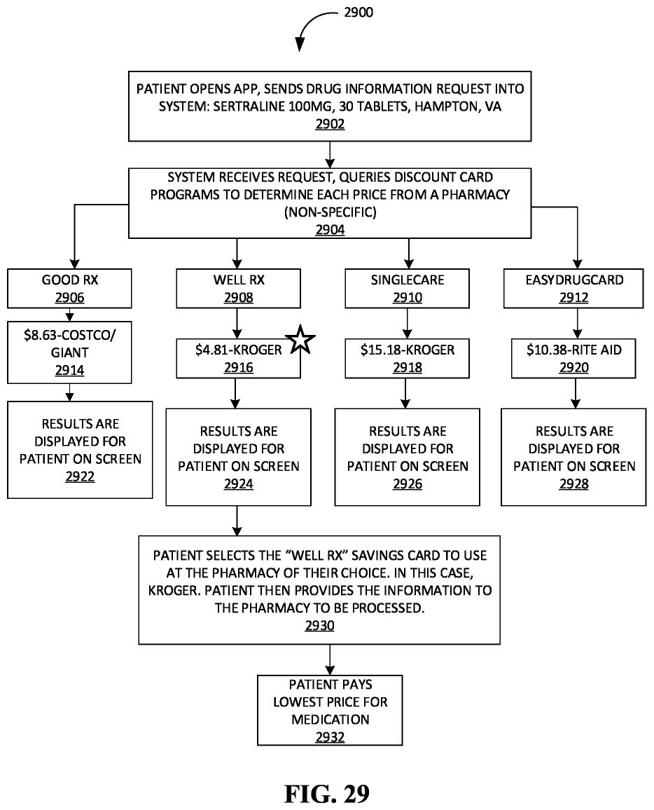

FIG. 29 is a flow diagram of a method 2900 for facilitating comparing prices of medications between different drug discount cards, in accordance with some embodiments. Accordingly, at 2902, the method 2900 may include a patient opening the software application and sending drug information request for medication. Further, at 2904, the method 2900 may include a system (such as the system 200) receiving requests and queries discount card programs. Further, the discount cards may include good RX 2906, Well RX 2908, singlecare 2910, and EasyDrug card 2912. Further, the good RX 2906 may be associated with a price 2914 of $8.63 at Costco/Giant pharmacy. Further, the Well RX 2908 may be associated with a price 2916 of $4.81 at Kroger. Further, the singlecare 2910 may be associated with a price 2918 of $15.18 at Kroger. Further, the EasyDrug card 2912 may be associated with a price 2920 of $10.38 at Rite aid. Further, at 2922, the method 2900 may include displaying the price 2914 for the patient on a screen. Further, at 2924, the method 2900 may include displaying the price 2916 for the patient on the screen. Further, at 2926, the method 2900 may include displaying the price 2918 for the patient on the screen. Further, at 2928, the method 2900 may include displaying the price 2920 for the patient on the screen. Further, at 2930, the method 2900 may include the patient selecting the Well RX card 2908 to use at a pharmacy of their choice, in an instance, the pharmacy may be Kroger. Further, at 2932, the method 2900 may include paying a lowest price for the medication.

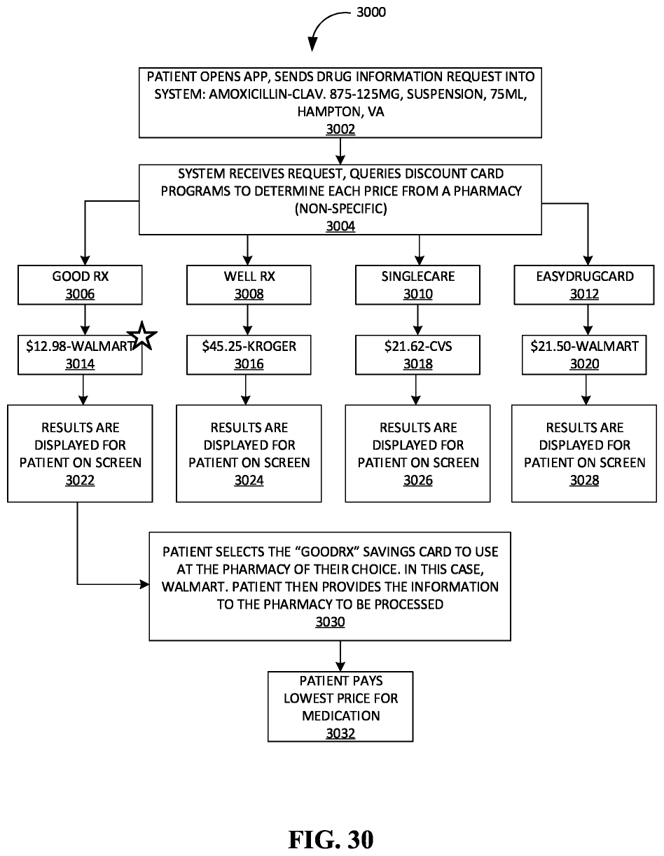

FIG. 30 is a flow diagram of a method 3000 for facilitating comparing prices of medications between different drug discount cards, in accordance with some embodiments.

Accordingly, at 3002, the method 3000 may include a patient opening the software application and sending drug information request for medication. Further, at 3004, the method 3000 may include a system (such as the system 200) receiving requests and queries discount card programs. Further, the discount cards may include good RX 3006, Well RX 3008, singlecare 3010, and EasyDrug card 3012. Further, the good RX 3006 may be associated with a price 3014 of $12.98 at Walmart. Further, the Well RX 3008 may be associated with a price 3016 of $45.25 at Kroger. Further, the singlecare 3010 may be associated with a price 3018 of $21.62 at CVS. Further, the EasyDrug card 3012 may be associated with a price 3020 of $21.50 at Walmart. Further, at 3022, the method 3000 may include displaying the price 3014 for the patient on a screen. Further, at 3024, the method 3000 may include displaying the price 3016 for the patient on the screen. Further, at 3026, the method 3000 may include displaying the price 3018 for the patient on the screen. Further, at 3028, the method 3000 may include displaying the price 3020 for the patient on the screen. Further, at 3030, the method 3000 may include the patient selecting the Good RX card 3006 to use at a pharmacy of their choice, in an instance, the pharmacy may be Walmart. Further, at 3032, the method 3000 may include paying a lowest price for the medication.

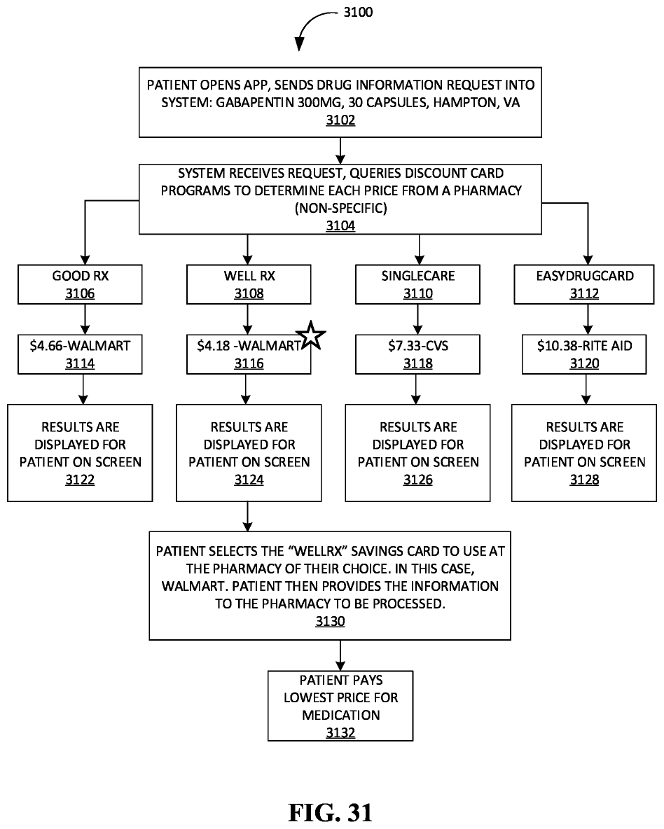

FIG. 31 is a flow diagram of a method 3100 for facilitating comparing prices of medications between different drug discount cards, in accordance with some embodiments. Accordingly, at 3102, the method 3100 may include a patient opening the software application and sending drug information request for medication. Further, at 3104, the method 3100 may include a system (such as the system 200) receiving requests and queries discount card programs. Further, the discount cards may include good RX 3106, Well RX 3108, singlecare 3110, and EasyDrug card 3112. Further, the good RX 3106 may be associated with a price 3114 of $4.66 at Walmart. Further, the Well RX 3108 may be associated with a price 3116 of $4.18 at Walmart. Further, the singlecare 3110 may be associated with a price 3118 of $7.33 at CVS. Further, the EasyDrug card 3112 may be associated with a price 3120 of $10.38 at Rite aid. Further, at 3122, the method 3100 may include displaying the price 3114 for the patient on a screen. Further, at 3124, the method 3100 may include displaying the price 3116 for the patient on the screen. Further, at 3126, the method 3100 may include displaying the price 3118 for the patient on the screen. Further, at 3128, the method 3100 may include displaying the price 3120 for the patient on the screen. Further, at 3130, the method 3100 may include the patient selecting the Well RX card 3108 to use at a pharmacy of their choice, in an instance, the pharmacy may be Walmart. Further, at 3132, the method 3100 may include paying a lowest price for the medication.

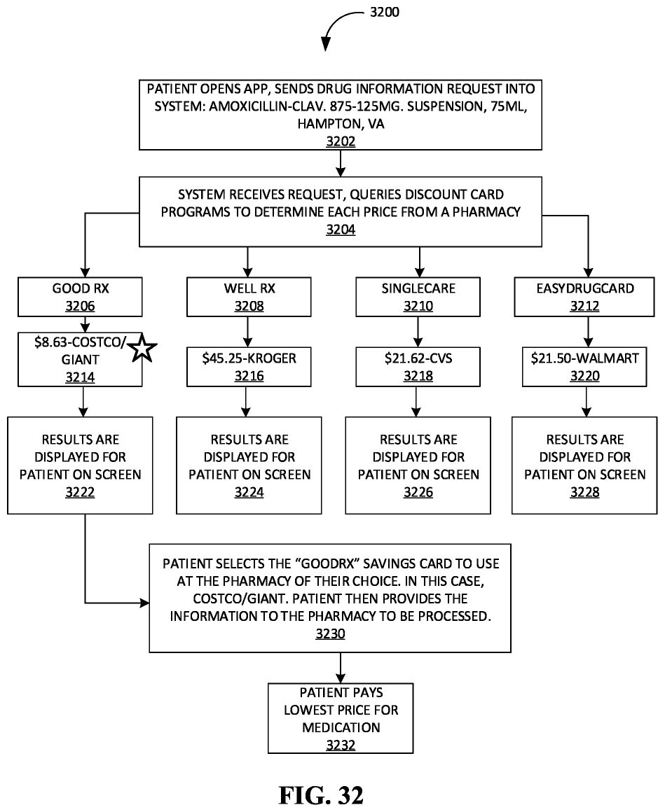

FIG. 32 is a flow diagram of a method 3200 for facilitating comparing prices of medications between different drug discount cards, in accordance with some embodiments. Accordingly, at 3202, the method 3200 may include a patient opening the software application and sending drug information request for medication. Further, at 3204, the method 3200 may include a system (such as the system 200) receiving requests and queries discount card programs. Further, the discount cards may include good RX 3206, Well RX 3208, singlecare 3210, and EasyDrug card 3212. Further, the good RX 3206 may be associated with a price 3214 of $8.63 at Costco/Giant. Further, the Well RX 3208 may be associated with a price 3216 of $45.25 at Kroger. Further, the singlecare 3210 may be associated with a price 3218 of $21.62 at CVS. Further, the EasyDrug card 3212 may be associated with a price 3220 of $21.50 at Walmart. Further, at 3222, the method 3200 may include displaying the price 3214 for the patient on a screen. Further, at 3224, the method 3200 may include displaying the price 3216 for the patient on the screen. Further, at 3226, the method 3200 may include displaying the price 3218 for the patient on the screen. Further, at 3228, the method 3200 may include displaying the price 3220 for the patient on the screen. Further, at 3230, the method 3200 may include the patient selecting the Good RX card 3206 to use at a pharmacy of their choice, in an instance, the pharmacy may be Costco/Giant. Further, at 3232, the method 3200 may include paying a lowest price for the medication.

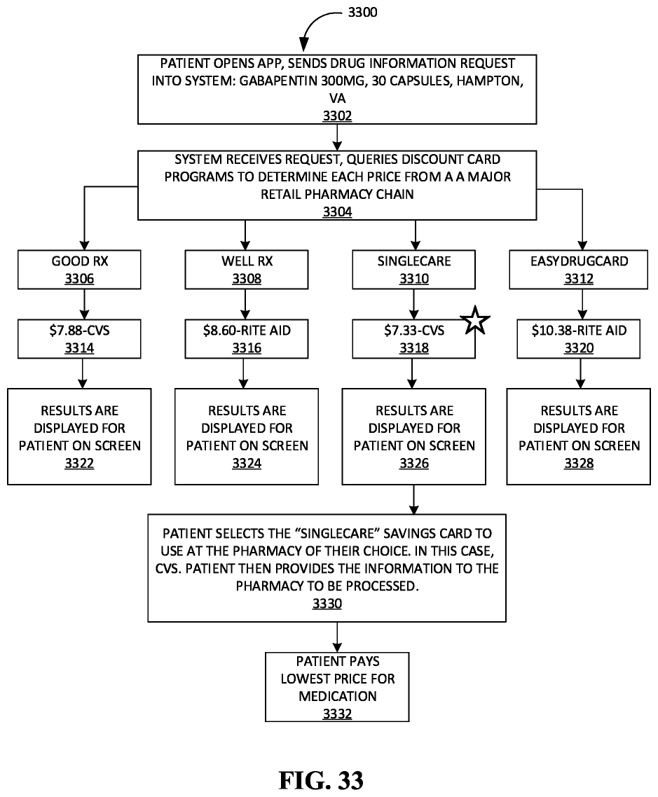

FIG. 33 is a flow diagram of a method 3300 for facilitating comparing prices of medications between different drug discount cards, in accordance with some embodiments. Accordingly, at 3302, the method 3300 may include a patient opening the software application and sending drug information request for medication. Further, at 3304, the method 3300 may include a system (such as the system 200) receiving requests and queries discount card programs. Further, the discount cards may include good RX (good RX card) 3306, Well RX (Well RX card) 3308, singlecare (or single care card) 3310, and EasyDrug card 3312. Further, the good RX 3306 may be associated with a price 3314 of $7.88 at CVS. Further, the Well RX 3308 may be associated with a price 3316 of $8.60 at Rite aid. Further, the singlecare card 3310 may be associated with a price 3318 of $7.33 at CVS. Further, the EasyDrug card 3312 may be associated with a price 3320 of $10.38 at Rite aid. Further, at 3322, the method 3300 may include displaying the price 3314 for the patient on a screen. Further, at 3324, the method 3300 may include displaying the price 3316 for the patient on the screen. Further, at 3326, the method 3300 may include displaying the price 3318 for the patient on the screen. Further, at 3328, the method 3300 may include displaying the price 3320 for the patient on the screen. Further, at 3330, the method 3300 may include the patient selecting the singlecare card 3310 to use at a pharmacy of their choice, in an instance, the pharmacy may be CVS. Further, at 3332, the method 3300 may include paying a lowest price for the medication.

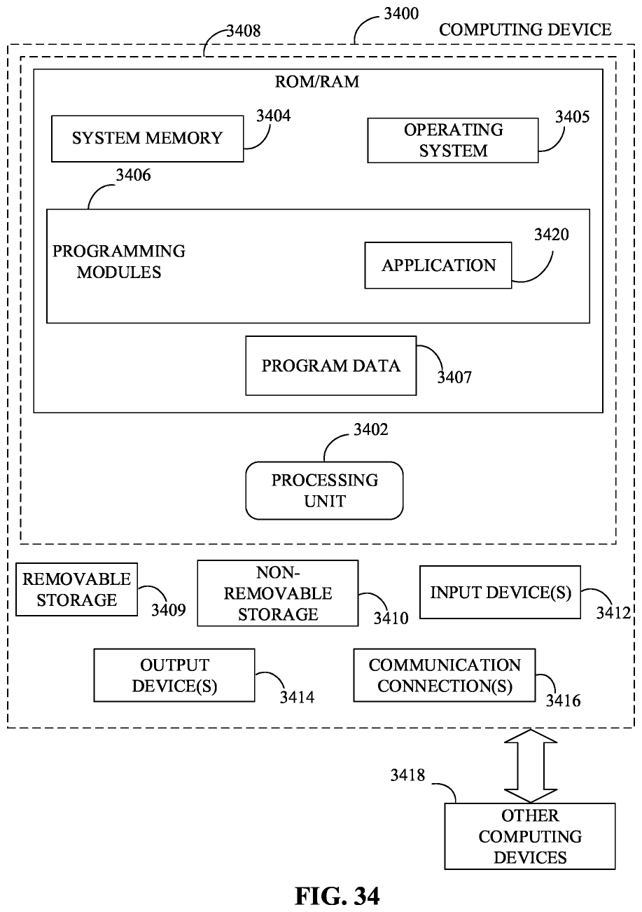

With reference to FIG. 34, a system consistent with an embodiment of the disclosure may include a computing device or cloud service, such as computing device 3400. In a basic configuration, computing device 3400 may include at least one processing unit 3402 and a system memory 3404. Depending on the configuration and type of computing device, system memory 3404 may comprise, but is not limited to, volatile (e.g. random-access memory (RAM)), non-volatile (e.g. read-only memory (ROM)), flash memory, or any combination. System memory 3404 may include operating system 3405, one or more programming modules 3406, and may include a program data 3407. Operating system 3405, for example, may be suitable for controlling computing device 3400's operation. In one embodiment, programming modules 3406 may include image-processing module, machine learning module. Furthermore, embodiments of the disclosure may be practiced in conjunction with a graphics library, other operating systems, or any other application program and is not limited to any particular application or system. This basic configuration is illustrated in FIG. 34 by those components within a dashed line 3408.

Computing device 3400 may have additional features or functionality. For example, computing device 3400 may also include additional data storage devices (removable and/or non-removable) such as, for example, magnetic disks, optical disks, or tape. Such additional storage is illustrated in FIG. 34 by a removable storage 3409 and a non-removable storage 3410. Computer storage media may include volatile and non-volatile, removable and non-removable media implemented in any method or technology for storage of information, such as computer-readable instructions, data structures, program modules, or other data. System memory 3404, removable storage 3409, and non-removable storage 3410 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 3400. Any such computer storage media may be part of device 3400. Computing device 3400 may also have input device(s) 3412 such as a keyboard, a mouse, a pen, a sound input device, a touch input device, a location sensor, a camera, a biometric sensor, etc. Output device(s) 3414 such as a display, speakers, a printer, etc. may also be included. The aforementioned devices are examples and others may be used.

Computing device 3400 may also contain a communication connection 3416 that may allow device 3400 to communicate with other computing devices 3418, such as over a network in a distributed computing environment, for example, an intranet or the Internet. Communication connection 3416 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 3404, including operating system 3405. While executing on processing unit 3402, programming modules 3406 (e.g., application 3420) may perform processes including, for example, one or more stages of methods, algorithms, systems, applications, servers, databases as described above. The aforementioned process is an example, and processing unit 3402 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

### REFERENCES

1. Kesselheim AS, Avorn J, Sarpatwari A. The high cost of prescription drugs in the

United States: Origins and prospects for reform. JAMA. 2016;316(8):858-871. [PubMed]

2. KFF (Kaiser Family Foundation). Kaiser health tracking poll: Health care priorities for 2017. 2017a. http://www.kff.org/health-costs/poll-finding/kaiser-health-tracking-poll-healthcare-priorities-for-2017.

3. KFF. Public opinion on prescription drugs and their prices. 2017b. http://www.kff.org/slideshow/public-opinion-on-prescription-drugs-and-their-prices.

4. Politico. Americans' top priorities for Congress through the end of the year. 2017. https://www.politico.com/f/?id=0000015e-7bce-d079-a3fe-7bce31540000.

The following is claimed:

1. A method for facilitating managing purchase of medications, the method comprising:
receiving, using a communication device, at least one medication request data associated with a patient from at least one device, wherein the at least one medication request data comprises at least one medication description of at least one medication and at least one patient identifier of the patient;
identifying, using a processing device, a plurality of stores based on the at least one medication description, wherein the plurality of stores provides the at least one medication;
receiving, using the communication device, a plurality of prices offered by the plurality of stores for the at least one medication from a plurality of store devices associated with the plurality of stores;
retrieving, using a storage device, at least one discount card data of at least one discount card associated with the patient based on the at least one patient identifier, wherein the at least one discount card data is used to reduce the plurality of prices offered by the plurality of stores for the at least one medication by at least one portion of the plurality of prices;
applying, using the processing device, the at least one discount card data to the plurality of prices offered by the plurality of stores for the at least one medication;
generating, using the processing device, a plurality of discounted prices offered by the plurality of stores for the at least one medication based on the applying;
retrieving, using the storage device, a plurality of store data associated with the plurality of stores based on the identifying; and
transmitting, using the communication device, the plurality of discounted prices offered by the plurality of stores for the at least one medication, the at least one discount card data, and the plurality of store data to the at least one device.

2. The method of claim 1, wherein the at least one medication request data comprises at least one preference data associated with at least one preference of the patient, wherein the method further comprises:
analyzing, using the processing device, the at least one preference data and the plurality of store data;
selecting, using the processing device, at least one store from the plurality of stores based on the analyzing; and
transmitting, using the communication device, at least one discounted price offered by the at least one store for the at least one medication and at least one store data of the at least one store to the at least one device.

3. The method of claim 2, wherein the at least one preference data comprises at least one location preference data associated with at least one location preference of the at least one preference, wherein the plurality of store data comprises a plurality of store location data associated with a plurality of store locations of the plurality of stores, wherein the analyzing comprises analyzing the at least one location preference data and the plurality of store location data, wherein the selecting of the at least one store is further based on the analyzing of the at least one location preference data and the plurality of store location data.

4. The method of claim 3, wherein the at least one device comprises a location sensor, wherein the location sensor is configured for generating patient location data based on a location of the patient, wherein the at least one location preference data comprises the patient location data.

5. The method of claim 4 further comprising:
analyzing, using the processing device, the patient location data and at least one store location data associated with the at least one store based on the selecting;
generating, using the processing device, at least one navigational direction from the location of the patient to at least one store location of the at least one store; and
transmitting, using the communication device, the at least one navigational direction to the at least one device.

6. The method of claim 2, wherein the at least one preference data comprises at least one purchase time preference data associated with at least one purchase time preference of the at least one preference, wherein the plurality of store data comprises a plurality of store operating duration data associated with a plurality of store operating durations of the plurality of stores, wherein the analyzing comprises analyzing the at least one purchase time preference data and the plurality of store operating duration data, wherein the selecting of the at least one store is further based on the analyzing of the at least one purchase time preference data and the plurality of store operating duration data.

7. The method of claim 1 further comprising:
analyzing, using the processing device, the plurality of discounted prices offered by the plurality of stores for the at least one medication;
determining, using the processing device, a lowest discounted price from the plurality of discounted prices based on the analyzing of the plurality of discounted prices;
identifying, using the processing device, a store offering the lowest discounted price from the plurality of stores based on the determining of the lowest discounted price; and
transmitting, using the communication device, the lowest discounted price offered by the store for the at least one medication and store data of the store to the at least one device.

8. The method of claim 1 further comprising:
receiving, using the communication device, the at least one discount card data from the at least one device, wherein the at least one device comprises at least one scanning device, wherein the at least one scanning device is configured for generating the at least one discount card data based on scanning at least one visual representation associated with the at least one discount card; and
storing, using the storage device, the at least one discount card data.

9. The method of claim 1, wherein the at least one discount card data comprises at least one redeeming restriction associated with a redeeming of the at least one discount card for the at least one medication at the plurality of stores, wherein the method further comprises:
analyzing, using the processing device, the at least one redeeming restriction with at least one of the at least one medication description and the plurality of store data using at least one machine learning model;
identifying, using the processing device, at least one redeemable discount card of the at least one discount card, a redeemable medication of the at least one medication, and at least one redeemable store of the plurality of stores based on the analyzing of the at least one redeeming restriction, wherein the at least one redeemable discount card is redeemable for the redeemable medication at the at least one redeemable store;
applying, using the processing device, at least one redeemable discount card data of the at least one redeemable discount card to at least one price offered by the at least one redeemable store for the redeemable medication;
determining, using the processing device, at least one discounted price offered by the at least one redeemable store for the redeemable medication based on the applying of the at least one redeemable discount card; and
transmitting, using the communication device, the at least one discounted price offered by the at least one redeemable store for the redeemable medication to the at least one device.

10. The method of claim 1 further comprising:
receiving, using the communication device, at least one store selection from the at least one device;
identifying, using the processing device, at least one store from the plurality of stores based on the at least one store selection;
receiving, using the communication device, at least one discount card selection from the at least one device;
identifying, using the processing device, at least one selected discount card from the at least one discount card based on the at least one discount card selection;
applying, using the processing device, at least one selected discount card data of the at least one selected discount card to at least one price offered by the at least one store for the at least one medication;
determining, using the processing device, at least one discounted price offered by the at least one store for the at least one medication based on the applying of the at least one first discount card;
generating, using the processing device, an order reference for the purchase of the at least one medication from the at least one store based on the determining of the at least one discounted price; and
transmitting, using the communication device, the order reference to the at least one device.

11. A system for facilitating managing purchase of medications, the system comprising:
a communication device configured for:
receiving at least one medication request data associated with a patient from at least one device, wherein the at least one medication request data comprises at least one medication description of at least one medication and at least one patient identifier of the patient;
receiving a plurality of prices offered by a plurality of stores for the at least one medication from a plurality of store devices associated with the plurality of stores; and
transmitting a plurality of discounted prices offered by the plurality of stores for the at least one medication, at least one discount card data, and a plurality of store data to the at least one device
a processing device communicatively coupled with the communication device, wherein the processing device is configured for:
identifying the plurality of stores based on the at least one medication description, wherein the plurality of stores provides the at least one medication;
applying the at least one discount card data to the plurality of prices offered by the plurality of stores for the at least one medication; and
generating the plurality of discounted prices offered by the plurality of stores for the at least one medication based on the applying; and
a storage device communicatively coupled with the processing device, wherein the storage device is configured for:
retrieving the at least one discount card data of at least one discount card associated with the patient based on the at least one patient identifier, wherein the at least one discount card data is used to reduce the plurality of prices offered by the plurality of stores for the at least one medication by at least one portion of the plurality of prices; and
retrieving the plurality of store data associated with the plurality of stores based on the identifying.

12. The system of claim 11, wherein the at least one medication request data comprises at least one preference data associated with at least one preference of the patient,
wherein the processing device is further configured for:
analyzing the at least one preference data and the plurality of store data; and
selecting at least one store from the plurality of stores based on the analyzing,
wherein the communication device is further configured for transmitting at least one discounted price offered by the at least one store for the at least one medication and at least one store data of the at least one store to the at least one device.

13. The system of claim 12, wherein the at least one preference data comprises at least one location preference data associated with at least one location preference of the at least one preference, wherein the plurality of store data comprises a plurality of store location data associated with a plurality of store locations of the plurality of stores, wherein the analyzing comprises analyzing the at least one location preference data and the plurality of store location data, wherein the selecting of the at least one store is further based on the analyzing of the at least one location preference data and the plurality of store location data.

14. The system of claim 13, wherein the at least one device comprises a location sensor, wherein the location sensor is configured for generating patient location data based on a location of the patient, wherein the at least one location preference data comprises the patient location data.

15. The system of claim 14, wherein the processing device is further configured for:
analyzing the patient location data and at least one store location data associated with the at least one store based on the selecting; and
generating at least one navigational direction from the location of the patient to at least one store location of the at least one store, wherein the communication device is further configured for transmitting the at least one navigational direction to the at least one device.

16. The system of claim 12, wherein the at least one preference data comprises at least one purchase time preference data associated with at least one purchase time preference of the at least one preference, wherein the plurality of store data comprises a plurality of store operating duration data associated with a plurality of store operating durations of the plurality of stores, wherein the analyzing comprises analyzing the at least one purchase time preference data and the plurality of store operating duration data, wherein the selecting of the at least one store is further based on the analyzing of the at least one purchase time preference data and the plurality of store operating duration data.

17. The system of claim 11, wherein the processing device is further configured for:
analyzing the plurality of discounted prices offered by the plurality of stores for the at least one medication;
determining a lowest discounted price from the plurality of discounted prices based on the analyzing of the plurality of discounted prices; and
identifying a store offering the lowest discounted price from the plurality of stores based on the determining of the lowest discounted price, wherein the communication device is further configured for transmitting the lowest discounted price offered by the store for the at least one medication and store data of the store to the at least one device.

18. The system of claim 11, wherein the communication device is further configured for receiving the at least one discount card data from the at least one device, wherein the at least one device comprises at least one scanning device, wherein the at least one scanning device is configured for generating the at least one discount card data based on scanning at least one visual representation associated with the at least one discount card, wherein the storage device is further configured for storing the at least one discount card data.

19. The system of claim 11, wherein the at least one discount card data comprises at least one redeeming restriction associated with a redeeming of the at least one discount card for the at least one medication at the plurality of stores, wherein the processing device is further configured for:
analyzing the at least one redeeming restriction with at least one of the at least one medication description and the plurality of store data using at least one machine learning model;
identifying at least one redeemable discount card of the at least one discount card, a redeemable medication of the at least one medication, and at least one redeemable store of the plurality of stores based on the analyzing of the at least one redeeming restriction, wherein the at least one redeemable discount card is redeemable for the redeemable medication at the at least one redeemable store;
applying at least one redeemable discount card data of the at least one redeemable discount card to at least one price offered by the at least one redeemable store for the redeemable medication; and
determining at least one discounted price offered by the at least one redeemable store for the redeemable medication based on the applying of the at least one redeemable discount card, wherein the communication device is further configured for transmitting the at least one discounted price offered by the at least one redeemable store for the redeemable medication to the at least one device.

20. The system of claim 11, wherein the communication device is further configured for:
receiving at least one store selection from the at least one device;
receiving at least one discount card selection from the at least one device; and
transmitting an order reference to the at least one device, wherein the processing device is further configured for:
identifying at least one store from the plurality of stores based on the at least one store selection;
identifying at least one selected discount card from the at least one discount card based on the at least one discount card selection;
applying at least one selected discount card data of the at least one selected discount card to at least one price offered by the at least one store for the at least one medication;
determining at least one discounted price offered by the at least one store for the at least one medication based on the applying of the at least one first discount card; and
generating the order reference for the purchase of the at least one medication from the at least one store based on the determining of the at least one discounted price.
